(19) United States
(12) Patent Application Publication
(10) Pub. No.: US 2013/0040897 A1
(43) Pub. Date: Feb. 14, 2013

(54) GLYCOSYLATION OF PROTEINS IN HOST CELLS

(71) Applicants: Markus Aebi, Wettingen (CH); Farnoush Parsaie Nasab, Zuerich (CH); Alexander Daniel Frey, Espoo (FI)

(72) Inventors: Markus Aebi, Wettingen (CH); Farnoush Parsaie Nasab, Zuerich (CH); Alexander Daniel Frey, Espoo (FI)

(73) Assignee: LONZA LTD., Visp (CH)

(21) Appl. No.: 13/643,018

(22) PCT Filed: Apr. 27, 2011
(86) PCT No.: PCT/EP2011/002094

§ 371 Date: Oct. 23, 2012

(30) Foreign Application Priority Data
Apr. 27, 2010 (EP) PCT/EP2010/002570

(51) Int. Cl. C12P 21/00; C12N 5/10; A61K 38/14; C12N 15/82; C12N 15/80; C07K 14/00; C12N 1/15; C12N 15/85

(52) U.S. Cl. 514/20.9; 435/254.11; 435/325; 435/419; 435/348; 435/455; 435/468; 435/471; 435/69.1; 530/395

(57) ABSTRACT

The invention provides means and methods for an improved production of glycosylated recombinant proteins in lower eukaryotes, specifically the production of human-like complex or hybrid glycosylated proteins in yeast. The invention provides genetically modified eukaryotic host cells capable of producing glycosylation optimized proteins useful as immunoglobulins and other therapeutic proteins, and provides cells capable of producing glycoproteins having glycan structures similar to glycoproteins produced in human cell. The invention further provides proteins with human-like glycan structures and novel compositions thereof producible by these modified cells.

### FIELD OF THE INVENTION

The invention relates to the field of glycoprotein production and protein glycosylation engineering in eukaryotes, specifically the production of human-like complex or hybrid glycosylated proteins in lower eukaryotes such as yeasts. The invention further relates to glycosylation modified eukaryotic host cells capable of producing glycosylation optimized proteins that are particularly useful as immunoglobulins and other therapeutic proteins for humans. The invention also relates to engineered eukaryotic, in particular non-human cells capable of producing glycoproteins having glycan structures similar to glycoproteins produced in human cells. Accordingly, the invention further relates to proteins with human-like glycan structures and novel compositions thereof that are producible by said cells.

### BACKGROUND OF THE INVENTION

The majority of protein-based biopharmaceuticals bare some form of post-translational modification which can profoundly affect protein properties relevant to their therapeutic application. Protein glycosylation represents the most common modification (about 50% of human proteins are glycosylated). Glycosylation can introduce considerable heterogeneity into a protein composition through the generation of different glycan structures on the proteins within the composition. Such glycan structures are made by the action of diverse enzymes of the glycosylation machinery as the glycoprotein transits the Endoplasmatic Reticulum (ER) and the Golgi-Complex (glycosylation cascade). The nature of the glycan structure(s) of a protein has impact on the protein's folding, stability, life time, trafficking, pharmacodynamics, pharmacokinetics and immunogenicity. The glycan structure often has great impact on the protein's primary functional activity. Glycosylation can affect local protein structure and may help to direct the folding of the polypeptide chain. One important kind of glycan structures are the so called N-glycans. They are generated by covalent linkage of an oligosaccharide to the amino (N)-group of asparagin residues in the consensus sequence NXS/T of the nascent polypeptide chain (N-glycosylation). N-glycans may further participate in the sorting or directing of a protein to its final target: the N-glycan of an antibody, for example, may interact with complement components. N-glycans also serve to stabilize a glycoprotein, for example, by enhancing its solubility, shielding hydrophobic patches on its surface, protecting from proteolysis, and directing intra-chain stabilizing interactions. Glycosylation may regulate protein half-life, for example, in humans the presence of terminal sialic acids in N-glycans may increase the half-life of proteins, circulating in the blood stream.

Such glycan structures are made by the action of several particular enzymes of the glycosylation machinery as the glycoprotein transits the endoplasmatic reticulum (ER) and the golgi-complex, both intracellular organelles represent the major components of the cellular glycosylation cascade. FIG. 1 depicts the LLO processing at the ER in wild type yeasts. Synthesis of the oligosaccharide occurs on both sides of the ER membrane. The glycosylation cascade starts with the generation of a lipid-linked oligosaccharide (LLO) on the cytosolic surface of the ER membrane. At first, a lipid-linked core oligosaccharide with a defined structure (Man3GlcNAc2) is synthesized. Further oligosaccharides are added onto the lipid dolichol-linked Man3GlcNAc2 on the cytosolic surface giving rise to the heptasaccharide Man5GlcNAc2 glycan structure. This LLO is then translocated (“flipped”) to the lumenal side of the ER. There further processing of the hepta-oligosaccharide chain to the branched oligosaccharide unit comprising three glucose, nine mannose, and two N-acetyl glucosamine residues (Glc3Man9GlcNAc2). A Glc3Man9GlcNAc2 structure is provided by the action of several glycosyl transferases. Each individual glycosyl transferase displays strong preference towards a certain oligosaccharide substrate. This leads to a basically linear, stepwise biosynthesis of the branched oligosaccharides. The Glc3Man9GlcNAc2 structure is then transferred from the dolichol lipid to the nascent polypeptide. Two steps of this ER glycosylation pathway are not directly related to the action of glycosyl transferases: (1) the flipping of the Man5GlcNAc2 LLO from the cytosolic side of the ER membrane to the lumenal side and (2) the oligosaccharyl transfer of the Glc3Man9GlcNAc2 glycan from the lipid-linker to the nascent polypeptide. LLO flipping is catalyzed by an ATP-independent bi-directional flippase. In yeast, the flippase activity is supported or conferred by “Rft1”, a polytopic membrane protein comprising about ten transmembrane domains, which span through the ER membrane. Genes for homologous proteins occur in the genomes of other eukaryotes.

Glycosyl transferases and glycosidases line the inner (lumenal) surface of the ER and Golgi apparatus and thereby provide a “catalytic” surface that allows for the sequential processing of glycoproteins as they proceed through the ER and through the Golgi network. In fact, the multiple compartments of the cis, medial, and trans Golgi and the trans-Golgi Network (TGN), provide the different localities in which the ordered sequence of glycosylation reactions can take place. As a glycoprotein proceeds from synthesis in the ER to full maturation in the late Golgi or TGN, it is sequentially exposed to the different glycosidases and glycosyl transferases along the glycosylation pathway. So the generated glycan structure of a protein is directly dependent on its individual contacts to the various enzymes of the glycosylation pathway. There might occur slight differences in such contacts between individual protein molecules which result in naturally occurring microheterogeneity in protein glycosylation.

The possibility of producing heterologous and/or recombinant proteins in host cells has revolutionized the treatment of patients with a variety of different diseases. Most therapeutic proteins need to be modified by the addition of glycan structures. This glycosylation may be necessary for correct folding, for long circulation and, in many cases, for optimal activity of the protein. Mammalian cells, like the commonly used Chinese hamster ovary cells (CHO cells) can produce complex glycan structures similar to human glycan structures, Nevertheless, glycan structures from e.g. CHO cells differ from glycan structures of human origin, as CHO cells a) sialylate at a lower degree, b) integrate additionally to the common sialic acid (NeuAc) another non-human sialic acid (NeuGc) and c) contain terminally bound α-1-3 galactose which is absent in human cells. Also the general pattern of glycan structures may differ in such a way that the relative amount of the complex GlcNAc2Man3GlcNAc2 glycan structure in comparison to further terminal galactosylated or sialylated glycans may be much higher in mammalian cell lines such as CHO cells than in non-old human cells. Disadvantages of the currently used mammalian expression systems for the production of recombinant proteins are (1) low productivity, (2) cost-intensive fermentation procedures, (3) need for complex strain design, (4) the risk of virus contamination, (5) a possibly non-complete human-like glycosylation, and (6) minimum possibilities to produce tailored glycosylation.

In contrast to such mammalian cells, yeast cells, for example, Pichia pastoris, Yarrowia lipolytica and Saccharomyces cerevisiae, are much more robust organisms for biotechnological production of recombinant proteins. Yeasts can be cultivated to high densities in well-defined media. However, glycosylation in yeast and fungi is very different from that in mammals and humans, although some common elements are shared: The first step of protein glycosylation, the transfer of the LLO to the nascent protein in the ER, is highly conserved in all eukaryotes including yeast, fungi, plants and humans. Subsequent processing of the obtained N-glycan in the Golgi, however, differs significantly between yeast and mammalian cells: In wild type yeast Golgi glycosylation mainly involves the addition of several mannose sugars. Such mannosylations are catalyzed by enzymatic action of mannosyl transferases residing in the Golgi, for example, Och1, Mnn1, Mnn2 and others.

The manufacture of therapeutic proteins with a reproducible and consistent glycoform profile remains a considerable challenge to the biopharmaceutical industry. In particular, therapeutic glycoproteins produced in yeast may trigger an unwanted immune response in higher eukaryotes, in particular animals and humans, leading to a low therapeutic value of therapeutic glycoproteins produced in yeast and the like. The impact of glycosylation on secretion, stability, immunogenicity and activity of several therapeutic proteins has been observed for several important therapeutic classes, including, blood factors, anticoagulants, thrombolytics, antibodies, hormones, stimulating factors and cytokines, for example, regulatory proteins of the TFN-family, EPO, gonadotropins, immunoglobulin G (IgG), granulocyte-macrophage colony-stimulating factor and interferons.

### SUMMARY OF THE INVENTION

It is the object of the present invention to provide means and methods for the production of glycosylated molecules such as lipids and proteins, in particular, recombinant glycoproteins, and as preferred examples glycoysylated immunoglobulins. It is a further object to provide a glycoprotein with a defined glycan structure, such as in particular a human-like or hybrid or complex glycan structure, and novel compositions thereof, that are producible by said means and methods. A particular object of the invention is the provision of N-glycosylated proteins and in particular immunoglobulins with a human-like glycan structure that are useable for therapy in humans with high therapeutic efficacy and without triggering unwanted side effects.

The technical problem underlying the present invention is primarily and fully solved by the provision of a novel genetically modified host cell. This cell is primarily characterized in that it is lacking or is having suppressed, diminished or depleted ER-localized glycosyl transferase activities, in particular mannosyl transferase activities. According to the invention the modified cell is lacking or is having suppressed, diminished or depleted ER-localized alpha-1,2-mannosyl transferase activity, more particular, Alg11-type activity. The cell is particularly characterized in that it is a knock-out mutant of the gene alg11 and/or of alg11 homologues. The cell is further lacking or is having suppressed, diminished or depleted ER-localized dolichyl phosphate-mannose glycolipid alpha-mannosyl transferase activity, more particular Alg3-type activity. The cell of is particularly characterized in that it is a also knock-out mutant of the gene alg3 and/or of alg3 homologues.

According to a particular embodiment of the invention the cell is further lacking or is having suppressed, diminished or depleted Golgi-localized mannosyl transferase activity, in particular Golgi-localized alpha-1,3-mannosyl transferase, more particular Mnn1-type activity. The cell of this particular embodiment is characterized in that it is also a knock-out mutant of the gene mnn1 and/or of mnn1 homologues.

According to the invention the cell of the invention is further genetically modified in the glycosylation pathway. The cell expresses, overexpresses or exhibits at least one or more heterologous glycosyl transferase activities. The nucleic acid molecule(s) may be present or included in the chromosome of the cell and/or form part of a expressable expression vector introduced in the cell.

More particular, the cell expresses, overexpresses one or more nucleic acid molecules coding for or exhibits mannosyl (alpha-1,3-)-glycoprotein beta-1,2-N-acetylglucosaminyl transferase (GnTI), i.e. GlcNAc transferase 1. More particular the cell expresses or overexpresses at least one nucleic acid molecule coding for GnTI or a catalytic domain thereof, for example the heterologous gene mgat I and/or homologues of mgat I.

In a particular variant thereof, the cell further expresses, overexpresses at least one or more nucleic acid molecules coding for or exhibits mannosyl (alpha-1,6-)-glycoprotein beta-1,2-N-acetylglucosaminyl transferase (GnTII), i.e. GlcNAc transferase 2. More particular the cell further expresses or overexpresses at least one nucleic acid molecule coding for GnTII or a catalytic domain thereof, for example the heterologous gene mgat II and/or homologues of mgat II.

In a particular variant thereof, the cell further expresses, overexpresses one or more nucleic acid molecules coding for or exhibits beta-N-acetylglucosaminyl glycopeptide beta-1,4-galactosyl transferase (GalT), i.e. Gal-transferase. More particular the cell further expresses or overexpresses at least one nucleic acid molecule coding for GalT or a catalytic domain thereof, for example the heterologous gene B4galT1 and/or homologues of B4galT1.

In an embodiment there is primarily provided a genetically modified host cell for the production of heterologous and/or recombinant glycosylated proteins, the cell having at least the following characteristics:
the cell is lacking or is depleted of ER-localized alpha-1,2-mannosyl transferase activity, in particular is depleted of alg11 and/or alg11 homologues or a knock out mutant thereof;
the cell is lacking or is depleted of ER-localized dolichyl phosphate-mannose glycolipid alpha-mannosyl transferase activity, in particular is depleted of alg3 and/or alg3 homologues or a knock out mutant thereof; and
the cell expresses or overexpresses heterologous mannosyl (alpha-1,3-)-glycoprotein beta-1,2-N-acetylglucosaminyl transferase (GnTI) activity.

In an alternative embodiment, there is primarily provided a genetically modified host cell for the production of heterologous and/or recombinant glycosylated proteins, the cell having at least the following characteristics:
the cell is lacking or is depleted of ER-localized alpha-1,2-mannosyl transferase activity, in particular is depleted of alg11 and/or alg11 homologues or a knock out mutant thereof;
the cell is lacking or is depleted of ER-localized beta-D-mannosyl transferase activity, in particular is depleted of dpm1 and/or dpm1 homologues or a knock out mutant thereof; and
the cell expresses or overexpresses heterologous mannosyl (alpha-1,3-)-glycoprotein beta-1,2-N-acetylglucosaminyl transferase (GnTI) activity.

In another alternative embodiment there is primarily provided a genetically modified host cell for the production of heterologous and/or recombinant glycosylated proteins, the cell having at least the following characteristics:
the cell is lacking or is depleted of ER-localized alpha-1,2-mannosyl transferase activity, in particular is depleted of alg11 and/or alg11 homologues or a knock out mutant thereof;
the cell is lacking or is depleted of ER-localized lipid linked monosaccharide (LLM) flippase activity, in particular is depleted of one or more genes encoding LLM flippase activity or a knock out mutant thereof; and
the cell expresses or overexpresses heterologous mannosyl (alpha-1,3-)-glycoprotein beta-1,2-N-acetylglucosaminyl transferase (GnTI) activity.

In more particular embodiments he cell of the invention is further characterized in that:
the cell is lacking or is depleted of Golgi-localized alpha-1,3 mannosyl transferase activity, in particular mnn1 or mnn1 homologue depleted or knock out.

As described in more detail herein below, the invention also provides methods and means to produce such modified cells. As also described in more detail herein below, the invention also provides methods and means for the production of glycosylated proteins in host cells as well as the glycosylated proteins produced according to the invention.

The cell may be further characterized in that it exhibits increased Rft1-type LLO flippase activity. The cell of this particular aspect is preferably further characterized in that the cell is overexpressing the gene rft1 or rft1 homologues.

The cell may be further characterized in that the cell expresses one or more further Golgi-localized heterologous enzyme or catalytic domain thereof, in particular selected from the group consisting of:
Mannosyl(beta-1,4-) glycoprotein-1,4-N-acetylglucosaminyl transferase (GnTIII); mannosyl (alpha-1,3-)-glycoprotein beta-1,4-N-acetylglucosaminyl transferase (GnTIV);
mannosyl (alpha-1,6-)-glycoprotein beta-1,6-N-acetylglucosaminyl transferase (GnTV);
mannosyl (alpha-1,6-)-glycoprotein beta-1,4-N-acetylglucosaminyl transferase (GnTVI);
alpha (1,6) fucosyl transferase (FucT);
beta-galactoside alpha-2,6-sialyl transferase (ST);
UDP-N-acetylglucosamine 2-epimerase (NeuC);
sialic acid synthase (NeuB);
CMP-Neu5Ac synthetase;
N-acylneuraminate-9-phosphate synthase;
N-acylneuraminate-9-phosphatase;
UDP-N-acetylglucosamine transporter;
UDP-galactose transporter;
GDP-fucose transporter;
CMP-sialic acid transporter;
nucleotide diphosphatase;
GDP-D-mannose 4,6-dehydratase; and
GDP-4-keto-6-deoxy-D-mannose-3,5-epimerase-4-reductase.

In a particular embodiment, this group of Golgi-localized heterologous enzymes may further include UDP-glucose 4-epimerase or UDP-galactose 4-epimerase-

In particular embodiments the present invention seeks to avoid the presence of any heterologous mannosidase activity in the cell, more particular the cell is lacking any heterologous enzyme activity of Golgi localized alpha-1,2-mannosidase or homologues thereof. In a particular embodiment the cell is lacking any heterologous mannosidase activity. In a particular variant the cell is lacking any mannosidase activity.

The cell is further characterized in that it is selected from: lower eukaryotic cells, including fungal cells, in particular yeast, and higher eukaryotic cells, including mammalian cells, plant cells, and insect cells.

In a further aspect there is provided a method for the production of a genetically modified cell, the method comprising at least the step(s) of: diminishing or depleting in the cell ER-localized alpha-1,2-mannosyl transferase activity (Alg 11); that is producing a knock-out mutant to alg11 and/or alg11 homologues; and the step(s) of diminishing or depleting in the cell ER-localized dolichyl phosphate-mannose glycolipid alpha-mannosyl transferase activity (Alg 3); that is producing a knock-out mutant to alg3 and/or a/g3 homologues. Accordingly, there is in particular provided a Δalg11Δalg3 knock out mutant strain.

In alternative embodiments of this aspect there is provided a method for the production of a genetically modified cell, the method comprising at least the step(s) of: diminishing or depleting in the cell ER-localized alpha-1,2-mannosyl transferase activity (Alg 11); that is producing a knock-out mutant to alg11 and/or alg11 homologues; and the step(s) of one or both of: diminishing or depleting in the cell ER-localized beta-D-mannosyl transferase activity (Dpm1), that is producing a knock-out mutant to dpm1 and/or dpm1 homologues, or diminishing or depleting in the cell ER-localized LLM flippase activity. Accordingly, there is in particular provided a Δalg11Δdpm1 knock out mutant strain and/or a Δalg11 LLM flippase knock out mutant strain.

In more particular embodiments the method further comprises the step(s) of: diminishing or depleting in the cell Golgi-localized alpha-1,3 mannosyl transferase activity (Mnn 1); that is producing an additional knock-out mutation to mnn1 and/or to mnn1 homologues. According to this embodiment, there is provided in particular a knock out mutant strain Δalg3Δalg11Δmnn1. In the respective alternative embodiment there is provided in particular a Δalg11Δdpm1 knock out mutant strain and/or a Δalg11 LLM flippase knock out mutant strain.

In a further embodiment thereof the method further comprises the step(s) of: transforming the cell with at least one nucleic acid molecule coding for heterologous mannosyl (alpha-1,3-)-glycoprotein beta-1,2-N-acetylglucosaminyl transferase (GnTI) activity, such that the cell is able to express or overexpress said activity.

In a further embodiment thereof the method further comprises the step(s) of: transforming the cell with at least one nucleic acid molecule coding for heterologous mannosyl (alpha-1,6-)-glycoprotein beta-1,2-N-acetylglucosaminyl transferase (GnTII) activity, such that the cell is able to express or overexpress said activity.

In a further embodiment thereof the method further comprises the step(s) of: transforming the cell with at least one nucleic acid molecule coding for heterologous beta-N-acetylglucosaminyl glycopeptide beta-1,4-galactosyl transferase (GalT) activity, such that the cell is able to express or overexpress said activity.

In a further embodiment thereof the method further comprises the step(s) of: transforming the cell with at least one nucleic acid molecule coding for an heterologous and/or recombinant protein as the substrate for glycosylation, such that the cell is able to express or overexpress said protein.

In more particular embodiments the method may include further steps of diminishing or depleting in the cell ER-localized and/or Golgi-localized mannosyl transferase activity or activity.

In more particular embodiments the method may include further steps of transforming the cell with one or more nucleic acid molecules coding for at least one further heterologous glycosyl transferase activity, such that the cell is able to express or overexpress said at least one further activity.

In a particular aspect, the invention provides a transformant host cell, specifically capable of producing one or more of the glycoprotein or glycoprotein compositions, in particular a recombinant protein, as characterized herein. As described in more detail herein below, the invention also provides host cells which may be further genetically modified to obtain particular strains that are specifically capable of producing a particular variant of glycosylated proteins with particular glycosylation pattern. The cell of the invention is thus further characterized in that it is modified to express or produce at least one heterologous and/or recombinant protein as substrate for glycosylation. The methods and means for producing cells for the production of heterologous and/or recombinant proteins of interest are well known in the art. The cell of the invention thus preferably comprises one or more nucleic acid molecules that code for one or more, in particular heterologous and/or recombinant, glycoproteins and is capable of producing the glycoprotein or compositions of one or more thereof.

The invention also provides the method or process to produce said glycoprotein or glycoprotein composition, wherein the method is primarily characterized in that the cell according the invention is provided and used to produce the glycoprotein.

The invention also provides glycoproteins, and in particular glycoprotein compositions, that are producible or are produced by the cell of the invention.

In a particular aspect, the invention provides a method for the production of a glycoprotein or a glycoprotein-composition, comprising the step(s) of:
providing a cell according to the invention;
culturing the cell in a culture medium under conditions that allow the production of the glycoprotein or glycoprotein-composition in said cell; and,
if necessary, isolating the glycoprotein or glycoprotein-composition from said cell and/or said culture medium.

In a further aspect, the invention provides a kit or kit-of-parts for producing glycoprotein, comprising:
the cell according to one of the preceding aspects of the invention and
culture medium for culturing the cell so as to confer the production of the glycoprotein.

The invention also provides an isolated or “substantially pure” nucleic acid molecule or a functional analog thereof, which is capable of encoding or conferring Rft1-type flippase activity in the ER.

The invention also provides an isolated or “substantially pure” nucleic acid molecule or a functional analog thereof, which is capable of encoding or conferring heterologous glycosyl transferase activity in the cell, in particular human GnT, human GalT and others, as described herein.

In a further aspect, the invention provides a glycoprotein or a glycoprotein composition, in particular a recombinant glycoprotein or a glycoprotein composition, characterized in that the glycan structure of the glycoprotein are selected from one or more of:
Man3GlcNAc2
Man4GlcNAc2
Man5GlcNAc2,
GlcNAcMan3GlcNAc2,
GlcNAcMan4GlcNAc2,
GlcNAcMan5GlcNAc2,
GlcNAc2Man3GlcNAc2,
GlcNAc3Man3GlcNAc2-bisecting
Gal1GlcNAc2Man3GlcNAc2,
Gal1GlcNAc2Man3GlcNAc2Fuc,
Gal1GlcNAc3Man3GlcNAc2-bisecting,
Gal1GlcNAc3Man3GlcNAc2Fuc-bisecting,
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc1Gal2GlcNAc2Man3GlcNAc2,
NeuAc1Gal2GlcNAc2Man3GlcNAc2Fuc,
NeuAc1Gal2GlcNAc3Man3GlcNAc2-bisecting,
NeuAc1Gal2GlcNAc3Man3GlcNAc2Fuc-bisecting,
NeuAc2Gal2GlcNAc2Man3GlcNAc2,
NeuAc2Gal2GlcNAc2Man3GlcNAc2Fuc,
NeuAc2Gal2GlcNAc3Man3GlcNAc2-bisecting,
NeuAc2Gal2GlcNAc3Man3GlcNAc2Fuc-bisecting,
GlcNAc3Man3GlcNAc2,
Gal1GlcNAc3Man3GlcNAc2,
Gal1GlcNAc3Man3GlcNAc2Fuc,
Gal2GlcNAc3Man3GlcNAc2,
Gal2GlcNAc3Man3GlcNAc2Fuc,
Gal3GlcNAc3Man3GlcNAc2,
Gal3GlcNAc3Man3GlcNAc2Fuc,
NeuAc1Gal3GlcNAc3Man3GlcNAc2,
NeuAc1Gal3GlcNAc3Man3GlcNAc2Fuc,
NeuAc2Gal3GlcNAc3Man3GlcNAc2,
NeuAc2Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

The invention is not limited to the production of glycoproteins with the above identified glycosylation structure.

In a further aspect, the invention provides a, particularly recombinant, glycoprotein, selected from:
glycoproteins, producible by the cell according to one of the preceding aspects of the invention,
glycoproteins, producible by the method according to one of the preceding aspects of the invention; and
glycoproteins according to the above identified aspect of the invention.

A preferred aspect thereof is a glycoprotein composition, comprising two or more of the glycoproteins according to this aspect. A preferred aspect thereof is a recombinant protein or a plurality thereof. A preferred aspect thereof is a therapeutically active protein or a plurality thereof. A preferred aspect thereof is an immunoglobulin or a plurality of immunoglobulins.

The desired glycan structure, in particular one or more of the above identified structures, may be present at the majority of the (recombinant) proteins produced, more particular at 60% or more, 70% or more, 80% or more, or 90% or more of the proteins.

In a further aspect, the invention provides a pharmaceutical composition, comprising: one or more of the glycoprotein of one of the preceding aspects of the invention and preferably at least one pharmaceutically acceptable carrier or adjuvant.

In yet a further aspect, the invention provides a method of treating a disorder that is treatable by administration of one or more of the glycoproteins or compositions of one or more of the preceding aspects, comprising the step(s) of: administering to a subject the glycoprotein or composition as described above, wherein the subject is suffering from, or is suspected to, a disease treatable by administration of that glycoprotein or composition.

### DETAILED DESCRIPTION OF THE INVENTION

The present invention primarily relates to host cells having modified lipid-linked oligosaccharides which may be modified further by heterologous expression of a set of glycosyl transferases and sugar transporters to become host-strains for the production of mammalian, e.g., human therapeutic glycoproteins. The process provides an engineered host cell which can be used to express and target any desirable gene(s) involved in glycosylation. Host cells with modified lipid-linked oligosaccharides are created or selected. N-glycans made in the engineered host cells primarily show a Man3GlcNAc2 core structure which may then be modified further by heterologous expression of one or more enzymes, e.g., glycosyl transferases and sugar transporters, to yield human-like glycoproteins. For the production of therapeutic proteins, this method may be adapted to engineer cell lines in which any desired glycosylation structure may be obtained (tailored glycosylation).

In some conditions it may be found that additional mannose residues are added afterwards in the Golgi apparatus by mannosyl transferases, which may result in Man4GlcNAc2 and Man5GlcNAc2 structures on the protein. In order to reduce the amount of the undesired Man4GlcNAc2 and Man5GlcNAc2 structures, the invention provides measures to avoid this. In a preferred aspect of the invention, the cell is thus further modified to lack or to have suppressed, diminished, or depleted one or more Golgi-localized glycosyl transferase activities, in particular mannosyl transferase activities, and in particular to express instead heterologous glycosyltransferase activities and other enzymes necessary for hybrid or complex N-glycosylation of proteins. The primary glycoprotein resulting from ER borne processing is subject to further glycosylation at the Golgi. The further major aspect of the present invention is a modification of the Golgi-based glycosylation. Modification of ER-based glycosylation and modification of the Golgi-based glycosylation go hand in hand to provide a system of combined modifications. For the first time the simple deletion of two ER-localized enzymes is combined with glycoengineering of the Golgi part of the glycosylation pathway (especially the heterologous expression of glycosyl transferases and deletion of endogenous mannosyl transferases at the Golgi). The present invention is in clear contrast to previous teachings of the prior art, wherein desired hypomannosylated glycans are obtained by trimming/cleavage of high-mannose (e.g. Man8GlcNAc2 or Man9GlcNAc2) or hypermannosylated glycoforms using homologous or heterologous mannosidase activities in one or both compartments of the glycosylation pathway (ER Golgi).

The cells according to the invention exhibit an increased ER-intralumenal concentration of Man3 type LLO in comparison to an unmodified wild type strain of the host cell. In particular, intralumenal concentration is increased by at least 5%, 10%, 15%, 20%, 25%, 30%, 40%, 50%, 70%, or 90%, more particular by at least 100%, 200%, 500%, 700%, 1000%, 1500%, 2000% or more, with respect to wild type cell. More particular, 85% or more, 90% or more, 95% or more of the ER-produced glycans in the modified host cell are of Man3 type.

For easy identification, all enzyme activities and genes described herein in connection with the present invention are primarily named according to their respective gene locus in the yeast S. cerevisiae. Although embodiments of the invention may concern yeast cells, in particular S. cerevisiae, the invention is not limited thereto. Modifications according to the invention may be applied to homologous structures in other cells or cell lines leading to the same effect as intended for the presently given working examples. The skilled person is able to identify respective activities present in other organisms, including prokaryotes, higher fungi and other eukaryotes. Examples of alternative cells and sources for heterologous enzyme activities are strains of Saccharomyces, Pichia, Yarrowia, Schizosaccharomyces, Klyveromyces, Aspergillus, Candida, and similar. Based on homologies amongst known enzymatic activities, one may, for example, design corresponding PCR primers or use genes or gene fragments encoding such enzymes as a probe to identify homologues in DNA and/or AA libraries of the target organism. Alternatively, one may be able to complement particular phenotypes in related organisms.

Alternatively, if the entire genomic sequence of a particular fungus of interest is known, one may identify such genes simply by searching publicly available DNA databases, which are available from several sources such as NCBI, Swissprot etc. For example, by searching a given genomic sequence or data base with a known gene from S. cerevisiae, one can identify genes of high homology in such a genome, which with a high degree of certainty encodes a gene that has a similar or identical activity. For example, homologues to known mannosyl transferases from S. cerevisiae in P. pastoris have been identified using either one of these approaches; these genes have similar functions to genes involved in the mannosylation of proteins in S. cerevisiae and thus their deletion may be used to manipulate the glycosylation pattern in P. pastoris or any other fungus with similar glycosylation pathways.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane Antibodies: A Laboratory Manual Cold Spring Harbor Laboratory Press, Cold Spring Harbor, N.Y. (1990); Introduction to Glycobiology, Maureen E. Taylor, Kurt Drickamer, Oxford Univ. Press (2003); Worthington Enzyme Manual, Worthington Biochemical Corp. Freehold, N.J.; Handbook of Biochemistry: Section A Proteins Vol I 1976 CRC Press; Handbook of Biochemistry: Section A Proteins Vol II 1976 CRC Press; Essentials of Glycobiology, Cold Spring Harbor Laboratory Press (1999). The nomenclatures used in connection with, and the laboratory procedures and techniques of, biochemistry and molecular biology described herein are those well known and commonly used in the art.

The present invention relates to genetically engineered cells where at least one endogenous enzyme activity is lacking or is being ineffective due one or more means, selected from suppression by inversion, suppression by antisense constructs, suppression by deletion, suppression on the level of transcription, suppression on the level of translation and other means. These are well known to a person skilled in molecular biology. In the context of the present invention by the term “knock-out” or “knock-out mutant” refers to both, full knock-out systems wherein the gene or transcript is not present at all, and partial knock-out mutants wherein the gene or transcript is still present but is silent or of little concentration, respectively, so that no considerable effect is exerted by the transcript in the cell.

The creation of gene knock-outs, once a given target gene sequence has been determined, is a well-established technique in the yeast and fungal molecular biology community, and can be carried out by anyone of ordinary skill in the art (e.g. see: R. Rothsteins, (1991) Methods in Enzymology, vol. 194, p. 281). In fact, the choice of a host organism may be influenced by the availability of good transformation and gene disruption techniques for such a host. If several transferases have to be knocked out, methods have been developed that allow for the repeated use of markers, for example, the URA3 markers to sequentially eliminate all undesirable endogenous transferase or other enzyme activity referred to herein. This technique has been refined by others but basically involves the use of two repeated DNA sequences, flanking a counter selectable marker. The presence of the marker is useful in the subsequent selection of transformants; for example, in yeast the ura3, his4, suc2, g418, bla, or shble genes may be used. For example, ura3 may be used as a marker to ensure the selection of a transformants that have integrated a construct. By flanking the ura3 marker with direct repeats one may first select for transformants that have integrated the construct and have thus disrupted the target gene. After isolation of the transformants, and their characterization, one may counter select in a second round for those that are resistant to 5′FOA. Colonies that are able to survive on plates containing 5′FOA have lost the ura3 marker again through a cross-over event involving the repeats mentioned earlier. This approach thus allows for the repeated use of the same marker and facilitates the disruption of multiple genes without requiring additional markers.

As used herein, the term “wild-type” as applied to a nucleic acid or polypeptide refers to a nucleic acid or a polypeptide that occurs in, or is produced by, respectively, a biological organism as that biological organism exists in nature.

The term “heterologous” as applied herein to a nucleic acid in a host cell or a polypeptide produced by a host cell refers to any nucleic acid or polypeptide (e.g., a protein having N-glycosylation activity) that is not derived from a cell of the same species as the host cell. Accordingly, as used herein, “homologous” nucleic acids, or proteins, are those that occur in, or are produced by, a cell of the same species as the host cell.

More particular, the term “heterologous” as used herein with reference to nucleic acid and a particular host cell refers to any nucleic acid that does not occur in (and cannot be obtained from) that particular cell as found in nature. Thus, a non-naturally-occurring nucleic acid is considered to be heterologous to a host cell once introduced into the host cell. It is important to note that non-naturally-occurring nucleic acids can contain nucleic acid subsequences or fragments of nucleic acid sequences that are found in nature provided that the nucleic acid as a whole does not exist in nature. For example, a nucleic acid molecule containing a genomic DNA sequence within an expression vector is non-naturally-occurring nucleic acid, and thus is heterologous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature.

It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be heterologous to a particular cell. For example, an entire chromosome isolated from a cell of yeast X is an heterologous nucleic acid with respect to a cell of yeast Y once that chromosome is introduced into a cell of yeast Y.

The terms “polynucleotide” or “nucleic acid molecule” refer to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruple, partially double-stranded, branched, hair-pinned, circular, or in a padlocked conformation. The term includes single and double stranded forms of DNA.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases, and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature.

The term “isolated” also can be used in reference to recombinant or cloned DNA isolates, chemically synthesized polynucleotide analogs, or polynucleotide analogs that are biologically synthesized by heterologous systems. However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g., by homologous recombination) for the native promoter of a gene in the genome of a human cell, such that this gene has an altered expression pattern. This gene would now become “isolated” because it is separated from at least some of the sequences that naturally flank it. A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced “artificially”, e.g., by human intervention. An “isolated nucleic acid” also includes a nucleic acid integrated into a host cell chromosome at a heterologous site, a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized.

The invention also provides respective means for direct genetic integration. The nucleotide sequence according to the invention, encoding the protein to be expressed in a cell may be placed either in an integrative vector or in a replicative vector (such as a replicating circular plasmid). Integrative vectors generally include serially arranged sequences of at least a first insertable DNA fragment, a selectable marker gene, and a second insertable DNA fragment. The first and second insertable DNA fragments are each about 200 nucleotides in length and have nucleotide sequences which are homologous to portions of the genomic DNA of the species to be transformed. A nucleotide sequence containing a structural gene of interest for expression is inserted in this vector between the first and second insertable DNA fragments whether before or after the marker gene. Integrative vectors can be linearized prior to yeast transformation to facilitate the integration of the nucleotide sequence of interest into the host cell genome.

As used herein, a “promoter” refers to a DNA sequence that enables a gene to be transcribed. The promoter is recognized by RNA polymerase, which then initiates transcription. A promoter contains a DNA sequence that is either bound directly by, or is involved in the recruitment, of RNA polymerase. A promoter sequence can also include “enhancer regions,” which are one or more regions of DNA that can be bound with proteins (namely, the trans-acting factors, much like a set of transcription factors) to enhance transcription levels of genes (hence the name) in a gene-cluster. The enhancer, while typically at the 5′ end of a coding region, can also be separate from a promoter sequence and can be, e.g., an intrinsic region of a gene or 3′ to the coding region of the gene.

According to the present invention the promoter is preferably the endogenous promoter of the gene. In a preferred embodiment the gene is on a high copy number plasmid which preferably leads to overexpression. In another preferred embodiment the gene is on a low copy number plasmid. The promoter may be a heterologous promoter. In a particular variant the promoter is a constitutive promoter. In another particular variant the promoter is an inducible promoter. A particular promoter according to the invention confers an overexpression of one or more copies of the nucleic acid molecule. In preferred embodiments, the molecule(s) is overexpressed two times, more preferred 5 times, 10 times, 20 times, 50 times, 100 times, 200 times, 500 times, 1000 times, and most preferred 2000 or more times when compared to expression from endogenous promoter. For example, where the host cell is Pichia pastoris, suitable promoters include, but are not limited to, aox1, aox2, das, gap, pex8, ypt1, fld1, and p40; where the host cell is Saccharomyces cerevisiae suitable promoters include, but are not limited to, gal1, mating factor a, cyc-1, pgk1, adh2, adh, tef, gpd, met25, galL, galS, ctr1, ctr3, and cup1. Where the host cell, for example, is a mammalian cell, suitable promoters include, but are not limited to CMV, SV40, actin promoter, rps21, Rous sarcoma virus genome large genome long terminal repeats (RSV), metallothionein, thymidine kinase or interferon gene promoter.

A “terminator” or 3′ termination sequences are able to the stop the transcription of a structural gene which function to stabilize the mRNA transcription product of the gene to which the sequence is operably linked, such as sequences which elicit polyadenylation. 3′ termination sequences can be obtained from Pichia or other methylotrophic yeast or other yeasts or higher fungi or other eukaryotic organisms. Examples of Pichia pastoris 3′ termination sequences useful for the practice of the present invention include termination sequences from the aox1 gene, p40 gene, his4 gene and fld1 gene.

According to the invention, there is also provided a vector for the transformation of a eukaryotic host cell, comprising one or more copies of one of the nucleic acid molecules characterized above or one or more copies of the expression cassette as characterized above.

The term “vector” as used herein is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial “artificial” chromosomes (BAC) and yeast “artificial” chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”).

The vectors of the present invention may contain a selectable marker gene. Examples of such systems include the Saccharomyces cerevisiae or Pichia pastoris his4 gene which may be used to complement his4 Pichia strains, or the S. cerevisiae or Pichia pastoris arg4 gene which may be used to complement Pichia pastoris arg mutants, or the Pichia pastoris ura3 and ade1 genes, which may be used to complement Pichia pastoris ura3 or ade1 mutants, respectively. Other selectable marker genes which function in Pichia pastoris include the zeo^{R} gene, the g418^{R} gene, blastisidin resistance gene, and the like. Maps of typical vectors useable according to the invention are schematically depicted in FIGS. 9 and 10.

The vectors of the present invention can also include an autonomous replication sequence (ARS). The vectors can also contain selectable marker genes which function in bacteria, as well as sequences responsible for replication and extrachromosomal maintenance in bacteria. In alternative embodiments the selection is conferred by auxothrophic markers. Examples of bacterial selectable marker genes include ampicillin resistance (amp^{r}), tetracycline resistance (tet^{r}), neomycin resistance, hygromycin resistance and zeocin resistance (zeo^{R}) genes.

A “host cell” according to the invention, is intended to relate to a cell into which a recombinant vector (e.g. expression vector) has been introduced or a linear recombinant DNA molecule has been integrated (e.g. chromosomal integration). It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. The term “cell” or “host cell” used for the production of a heterologous glycoprotein refers to a cell into which a nucleic acid, e.g. encoding a heterologous glycoprotein, can be or is introduced/transfected. Such cells include both prokaryotic cells, which are used for propagation of vectors/plasmids, and eukaryotic cells.

In particular embodiments, the host cell is a mammalian cell. In variants, the cell is selected from, preferably immortalized, cell lines such as hybridoma cells, myeloma cells, for example, rat myeloma cells and mouse myeloma cells, or human cells. In variants thereof the cell is selected from, but not limited to, CHO cells, in particular CHO K-1 and CHO DG44, BHK cells, NSO cells, SP2/0 cells, HEK293 cells, HEK293-EBNA cells, PER.C6 cells, COS cells, 3T3 cells, YB2 cells, HeLa cells, and Vero cells. In particular variants the cell is selected from DHFR-deficient CHO cells, such as dhfr⁻CHO (Proc. Natl. Acad. Sci. USA, Vol. 77, p. 4216-4220, 1980) and CHO K-1 (Proc. Natl. Acad. Sci. USA, Vol. 60, p. 1275, 1968).

In other embodiments, the host cell is an amphibian cell. Preferably, the cell is selected from, but not limited to, Xenopus laevis oocytes (Nature, Vol. 291, p. 358-360, 1981).

In other embodiments, the host cell is an insect cell. Preferably, the cell is selected from, but not limited to, Sf9, Sf21, and Tn5.

In other embodiments, the host cell is a plant cell. Preferably, the cell is selected from, but not limited to, cells derived from Nicotiana tabacum, the acquatic plant Lemna minor or the moss Physcomitrella patens. These cells are known as a system for producing polypeptides, and may be cultured also as calli.

In preferred embodiments, the host cell is a lower eukaryotic cell. Lower eukaryotic cells according to the invention include, but are not limited to, unicellular, multicellular, and filamentous fungi, preferably selected from: Pichia sp. Candida sp. Saccharomyces sp., Saccharomycodes sp., Saccharomycopsis sp., Schizosaccharomyces sp., Zygosaccharomyces sp. Yarrowia sp., Hansenula sp., Kluyveromyces sp., Trichoderma sp, Aspergillus sp., and Fusarium sp. and Myceteae, preferably selected from Ascomycetes, in particular Chysosporium lucknowense, and Basidiomycetes, in particular Coniphora sp. as well as Arxula sp.

In more preferred variants thereof the cell is selected from, but not limited to: P. pastoris, P. stiptis, P. methanolica, P. bovis, P. canadensis, P. fermentans, P. membranaefaciens, P. pseudopolymorpha, P. quercuum, P. robertsii, P. saitoi, P. silvestrisi, P. strasburgensis; P. finlandica, P. trehalophila, P. koclamae, P. opuntiae, P. thermotolerans, P. salictaria, P. guercuum, P. pijperi; C. albicans, C. amphixiae, C. atlantica, C. corydalis, C. dosseyi, C. fructus, C. glabrata, C. fermentati, C. krusei, C. lusitaniae, C. maltosa, C. membranifaciens, C. utilis; S. bayanus, S. cerevisiae, S. bisporus, S. delbrueckii, S. fermentati, S. fragilis, S. mellis, S. rosei; Saccharomycodes ludwigii, Saccharomycopsis capsularis; Schizosaccharomyces pombe, Schizosaccharomyces octosporus, Zygosaccharomyces bisporus, Zygosaccharomyces mellis, Zygosaccharomyces rouxii; Yarrowia lipolytica, Hansenula polymorpha, Kluyveromyces sp., Trichoderma reseei, A. nidulans, A. candidus, A. carneus, A. clavatus, A. fumigatus, A. niger, A. oryzae, A. versicolor, Fusarium gramineum, Fusarium venenatum, and Neurospora crassa as well as Arxula adeninivorans.

In particular embodiments the cell exhibits a further modified ER-based glycosylation processing. More particular, one or more further enzyme activity conferring glycosylation, in particular manosylation in the ER are diminished or depleted in the cell, in particular by knock-out mutation of one or more genes coding for that enzyme activity. The invention is not limited to such knock-out mutants of ER-glycosylation.

In a variant there is provided a alg11 depleted or Δalg11 knock-out mutant strain which is further lacking or is having suppressed, diminished or depleted one or more dolichyl-phosphate beta-D-mannosyl transferase type activity, in particular is also depleted or a knock-out mutant for dpm1 and/or dpm1 homologues. In another variant there is provided a a/g3 alg11 depleted or Δalg3Δalg1/knock-out mutant strain which is further lacking or is having suppressed, diminished or depleted one or more lipid-linked monosaccharide (LLM) flippase type activities in particular is also depleted or a knock-out mutant for one or more genes coding for lipid-linked monosaccharide (LLM) flippase activity.

In an alternative variant there is provided a alg11, mnn1 depleted or Δalg11Δmnn1 knock-out mutant strain which is further lacking or is having suppressed, diminished or depleted one or more dolichyl-phosphate beta-D-mannosyl transferase type activity, in particular is also depleted or a knock-out mutant for dpm1 and/or dpm1 homologues. In another alternative variant there is provided a alg11, mnn1 depleted or Δalg11Δmnn1 knock-out mutant strain which is further lacking or is having suppressed, diminished or depleted one or more lipid-linked monosaccharide (LLM) flippase type activities in particular is also depleted or a knock-out mutant for one or more genes coding for lipid-linked monosaccharide (LLM) flippase activity.

The primary glycoprotein resulting from oligosaccharyl transferase activity at the ER may be subject to further glycosylation at the Golgi as described below in more detail. The further major aspect of the present invention is the provision of means and methods for the modification of the Golgi-based glycosylation in the host cell of the invention. Modification of ER-based glycosylation as described in more detail hereinabove and modification of the Golgi-based glycosylation as described in more detail herein, go hand in hand. This invention advantageously provides primary glycoproteins with low-mannose glycan structure which form the ideal substrate for the subsequent modified glycosylation in the Golgi.

In preferred embodiments the host cell is further modified or genetically engineered to lack or be diminished or depleted in one more, at least two more, preferably at least three more, at least four more or at least five more Golgi-localized mannosyl transferases. Although the invention is primarily directed to N-glycosylation, it also optionally foresees the diminishing or depletion of one or more mannosyl transferases of the O-glycosylation pathway. It has been found that mannosyl transferases of the O-glycosylation pathway may exhibit some transferase activity also in the N-glycosylation pathway.

The mannosyl transferases are preferably selected from Table 1 and homologues thereof. Accordingly, particular variants of the cell of the invention may be a knock-out mutant of at least one gene selected from: och1, hoc1, mnn2, mnn5, mnn6, ktr6, mnn8, anp1, mnn9, mnn10, mnn11, mnt1, kre2, mnt2, mnt3, mnt4, ktr1, ktr2, ktr3, ktr4, ktr5, ktr7, van1, and yur1, and the homologues thereof. Homologues also include other members of the same or a related gene family. The invention is not limited to these knock-out variants.

In a first variant of an embodiment there is provided a Δalg36,alg11Δmnn1 depleted or knock-out mutant strain which is also depleted or a knock-out mutant for och1. In another variant there is provided a Δalg3Δalg11Δmnn1 depleted or knock-out mutant strain which is also depleted or a knock-out mutant for hoc1. In another variant there is provided a Δalg3Δalg11Δmnn1 depleted or knock-out mutant strain which is also depleted or a knock-out mutant for mnn2. In another variant there is provided a Δalg3Δalg11Δmnn1 depleted or knock-out mutant strain which is also depleted or a knock-out mutant for mnn3. In another variant there is provided a Δalg3Δalg11Δmnn1 depleted or knock-out mutant strain which is also depleted or a knock-out mutant for mnn5. In another variant there is provided a Δalg3Δalg111 nm n1 depleted or knock-out mutant strain which is also depleted or a knock-out mutant for mnn6/ktr6. In another variant there is provided a Δalg3Δalg11Δmnn1 depleted or knock-out mutant strain which is also depleted or a knock-out mutant for mnn8/anp1. In another variant there is provided a Δalg3Δalg11Δmnn1 depleted or knock-out mutant strain which is also depleted or a knock-out mutant for mnn9. In another variant there is provided a Δalg3Δalg11Δmnn1 depleted or knock-out mutant strain which is also depleted or a knock-out mutant for mnn10. In another variant there is provided a Δalg3Δalg11Δmnn1 depleted or knock-out mutant strain which is also depleted or a knock-out mutant for mnn11. In another variant there is provided a Δalg3Δalg11Δmnn1 depleted or knock-out mutant strain which is also depleted or a knock-out mutant for mnt1/kre2. In another variant there is provided a Δalg3Δalg11Δmnn1 depleted or knock-out mutant strain which is also depleted or a knock-out mutant for mnt2. In another variant there is provided a Δalg36,alg11Δmnn1 depleted or knock-out mutant strain which is also depleted or a knock-out mutant for mnt3. In another variant there is provided a Δalg36,alg11Δmnn1 depleted or knock-out mutant strain which is also depleted or a knock-out mutant for mnt4. In another variant there is provided a Δalg36,alg11Δmnn1 depleted or knock-out mutant strain which is also depleted or a knock-out mutant for ktr1. In another variant there is provided a Δalg3Δalg11Δmnn1 depleted or knock-out mutant strain which is also depleted or a knock-out mutant for ktr2. In another variant there is provided a Δalg3Δalg11Δmnn1 depleted or knock-out mutant strain which is also depleted or a knock-out mutant for ktr3. In another variant there is provided a Δalg3Δalg11Δmnn1 depleted or knock-out mutant strain which is also depleted or a knock-out mutant for ktr4. In another variant there is provided a Δalg36,alg11Δmnn1 depleted or knock-out mutant strain which is also depleted or a knock-out mutant for ktr5. In another variant there is provided a Δalg3Δalg11Δmnn1 depleted or knock-out mutant strain which is also depleted or a knock-out mutant for ktr7. In another variant there is provided a Δalg36,alg11Δmnn1 depleted or knock-out mutant strain which is also depleted or a knock-out mutant for van1. In another variant there is provided a Δalg36,alg11Δmnn1 depleted or knock-out mutant strain which is also depleted or a knock-out mutant for yur1.

In a first variant of another embodiment the invention there is provided a Δalg3Δalg11 depleted or knock-out mutant strain which is also depleted or a knock-out mutant for och1. In another variant there is provided a Δalg3Δalg11 depleted or knock-out mutant strain which is also depleted or a knock-out mutant for hoc1. In another variant there is provided a Δalg36,alg11 depleted or knock-out mutant strain which is also depleted or a knock-out mutant for mnn2. In another variant there is provided a Δalg3Δalg11 depleted or knock-out mutant strain which is also depleted or a knock-out mutant for mnn3. In another variant there is provided a Δalg3Δalg11 depleted or knock-out mutant strain which is also depleted or a knock-out mutant for mnn5. In another variant there is provided a Δalg3Δalg11 depleted or knock-out mutant strain which is also depleted or a knock-out mutant for mnn6/ktr6. In another variant there is provided a Δalg3Δalg11 depleted or knock-out mutant strain which is also depleted or a knock-out mutant for mnn8/anp1. In another variant there is provided a Δalg3Δalg11 depleted or knock-out mutant strain which is also depleted or a knock-out mutant for mnn9. In another variant there is provided a Δalg3Δalg11 depleted or knock-out mutant strain which is also depleted or a knock-out mutant for mnn10. In another variant there is provided a Δalg3Δalg11 depleted or knock-out mutant strain which is also depleted or a knock-out mutant for mnn11. In another variant there is provided a Δalg3Δalg11 depleted or knock-out mutant strain which is also depleted or a knock-out mutant for mnt1/kre2. In another variant there is provided a Δalg3Δalg11 depleted or knock-out mutant strain which is also depleted or a knock-out mutant for mnt2. In another variant there is provided a Δalg3Δalg11 depleted or knock-out mutant strain which is also depleted or a knock-out mutant for mnt3. In another variant there is provided a Δalg3Δalg11 depleted or knock-out mutant strain which is also depleted or a knock-out mutant for mnt4. In another variant there is provided a Δalg3Δalg1/depleted or knock-out mutant strain which is also depleted or a knock-out mutant for ktr1. In another variant there is provided a Δalg36,alg11 depleted or knock-out mutant strain which is also depleted or a knock-out mutant for ktr2. In another variant there is provided a Δalg3Δalg11 depleted or knock-out mutant strain which is also depleted or a knock-out mutant for ktr3. In another variant there is provided a Δalg3Δalg11 depleted or knock-out mutant strain which is also depleted or a knock-out mutant for ktr4. In another variant there is provided a Δalg3Δalg11 depleted or knock-out mutant strain which is also depleted or a knock-out mutant for ktr5. In another variant there is provided a Δalg3Δalg11 depleted or knock-out mutant strain which is also depleted or a knock-out mutant for ktr7. In another variant there is provided a Δalg3Δalg11 depleted or knock-out mutant strain which is also depleted or a knock-out mutant for van1. In another variant there is provided a Δalg3Δalg11 depleted or knock-out mutant strain which is also depleted or a knock-out mutant for yur1.

TABLE 1
Golgi-localized mannosyl transferases

| | | Synonymous or |
|---|---|---|
| Name | Function | systematic name |
| Och1 | alpha-1,6-mannosyl transferase | YGL048C |
| Hoc1 | alpha-1,6-mannosyl transferase | YJR075W |
| Mnn1 | alpha-1,3-mannosyl transferase | YER001W |
| Mnn2 | alpha1,2-mannosyl transferase | YBR015C, TTP1, |
| | | CRV4, LDB8 |
| Mnn5 | alpha1,2-mannosyl transferase | YJL186W |
| Mnn6 | mannosylphosphate transferase | YPL053C |
| (Ktr6) | | |
| Mnn8 | alpha-1,6 mannosyl transferase | YEL036C |
| (Anp1) | | |
| Mnn9 | Subunit of a Golgi mannosyltransferase | YPL050C |
| Mnn10 | Subunit of a Golgi mannosyltransferase | YDR245W, BED1, |
| | | SLC2, REC41 |
| Mnn11 | Subunit of a Golgi mannosyltransferase | YJL183W |
| Mnt1 | alpha-1,2-mannosyl transferase | YDR483W |
| (Kre2) | | |
| Mnt2 | alpha-1,3-mannosyl transferase | YGL257C |
| Mnt3 | alpha-1,3-mannosyl transferase | YIL014W |
| Mnt4 | alpha-1,3-mannosyl transferase | YNR059W |
| Ktr1 | alpha-1,2-mannosyltransferase | YOR099W |
| Ktr2 | Mannosyl transferase | YKR061W |
| Ktr3 | Putative alpha-1,2-mannosyl transferase | YBR205W |
| Ktr4 | Putative mannosyl transferase | YBR199W |
| Ktr5 | Putative mannosyl transferase | YNL029C |
| Ktr7 | Putative mannosyl transferase | YIL085C |
| Van1 | Component of the mannan polymerase I | YML115C |
| Yur1 | Golgi mannosyl transferase | YJL139C |

The cell of the invention may be further genetically engineered to alter the glycosylation cascade, in particular within the Golgi. The invention provides a cell capable of the production of a glycoprotein or glycoprotein composition that exhibit a certain type of N-glycan structure such as, for example, a human-like glycan structure in a cell other than a human cell. Accordingly, such cell may be genetically further modified in the Golgi glycosylation pathway that allow the cell to carry out a sequence of enzymatic reactions, which mimic the processing of glycoproteins in e.g. humans. Recombinant proteins expressed in these engineered cells yield glycoproteins more similar, if not substantially identical, to their human counterparts. Embodiments include, but are not limited to, recombinant glycoproteins comprising one or more of glycan structure selected from:
Man3GlcNAc2
Man4GlcNAc2
Man5GlcNAc2,
GlcNAcMan3-5GlcNAc2,
GlcNAc2Man3GlcNAc2,
GlcNAc3Man3GlcNAc2-bisecting
Gal1GlcNAc2Man3GlcNAc2,
Gal1GlcNAc2Man3GlcNAc2Fuc,
Gal1GlcNAc3Man3GlcNAc2-bisecting,
Gal1GlcNAc3Man3GlcNAc2Fuc-bisecting,
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc1Gal2GlcNAc2Man3GlcNAc2,
NeuAc1Gal2GlcNAc2Man3GlcNAc2Fuc,
NeuAc1Gal2GlcNAc3Man3GlcNAc2-bisecting,
NeuAc1Gal2GlcNAc3Man3GlcNAc2Fuc-bisecting,
NeuAc2Gal2GlcNAc2Man3GlcNAc2,
NeuAc2Gal2GlcNAc2Man3GlcNAc2Fuc,
NeuAc2Gal2GlcNAc3Man3GlcNAc2-bisecting,
NeuAc2Gal2GlcNAc3Man3GlcNAc2Fuc-bisecting,
GlcNAc3Man3GlcNAc2,
Gal1GlcNAc3Man3GlcNAc2,
Gal1GlcNAc3Man3GlcNAc2Fuc,
Gal2GlcNAc3Man3GlcNAc2,
Gal2GlcNAc3Man3GlcNAc2Fuc,
Gal3GlcNAc3Man3GlcNAc2,
Gal3GlcNAc3Man3GlcNAc2Fuc,
NeuAc1Gal3GlcNAc3Man3GlcNAc2,
NeuAc1Gal3GlcNAc3Man3GlcNAc2Fuc,
NeuAc2Gal3GlcNAc3Man3GlcNAc2,
NeuAc2Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

More particular embodiments include recombinant glycoproteins comprising one or more of glycan structure selected from:
GlcNAcMan3-5GlcNAc2,
GlcNAc2Man3GlcNAc2,
GlcNAc3Man3GlcNAc2-bisecting
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc2Gal2GlcNAc2Man3GlcNAc2,
NeuAc2Gal2GlcNAc2Man3GlcNAc2Fuc,
NeuAc2Gal2GlcNAc3Man3GlcNAc2-bisecting,
euAc2Gal2GlcNAc3Man3GlcNAc2Fuc-bisecting,
GlcNAc3Man3GlcNAc2,
Gal3GlcNAc3Man3GlcNAc2,
Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

As used herein GlcNAc is N-acetylglucosamine, Gal is galactose, Fuc is fucose, and NeuAc is N-acetylneuraminic acid, i.e. sialic acid. As used herein, in preferred embodiments all glycan structures lack fucose in their glycan structures unless the presence of fucose (Fuc) is specifically exemplified.

According to the present invention this is preferably achieved by engineering and/or selection of strains which lack certain enzyme activities that create undesirable high mannose type structures characteristic of glycoproteins of lower eukaryotes, in particular fungal cells such as yeasts. This is preferably achieved by engineering host cells which express heterologous activities which generate glycan structures which are not recognized by enzymes creating the high mannose type, which are selected either to have optimal activity under the conditions present in the lower eukaryotic cell such as a fungi where activity is desired, or which are targeted to an organelle where optimal activity is achieved, and combinations thereof wherein the genetically engineered eukaryote expresses multiple heterologous enzymes required to produce “human-like” glycoproteins.

In preferred embodiments the present invention also concerns the integration of one or more heterologous enzyme activities in the Golgi that are capable of producing “human-like” N-glycans. In preferred embodiments, the invention provides genetically engineered cells which comprise in the Golgi at least one heterologous glycosyl transferase activity and/or one or more glycosyl transferase activity associated activity selected from the group of activities listed in Tables 2, 3, and 4.

Human-like glycosylation is primarily characterized by “complex” N-glycan structures containing N-acetylglucosamine, galactose, fucose and/or N-acetylneuraminic acid. Other sialic acids like N-glycolylneuraminic acid present in N-glycans from other mammals like hamster are absent in humans. Also special oligosaccharyl linkages like terminally bound alpha-1-3 galactose is typical for rodents but absent in human cells.

TABLE 2
Heterologous glycosyl transferases, transporters and associated enzymes

| | Function/enzymatic | primary | | | Gene |
|---|---|---|---|---|---|
| Name | activity | Location | E.C. | Synonymous name(s) | name |
| GnTI | mannosyl (alpha-1,3-)- | Golgi | 2.4.1.101 | GlcNAc transferase 1, | Mgat1 |
| | glycoprotein beta-1,2- | | | alpha-1,3-mannosyl- | |
| | N-acetylglucosaminyl | | | glycoprotein beta-1,2-N- | |
| | transferase | | | acetylglucosaminyl | |
| | | | | transferase | |
| GnTII | mannosyl (alpha-1,6-)- | Golgi | 2.4.1.143 | GlcNAc transferase 2, | Mgat2 |
| | glycoprotein beta-1,2- | | | N-acetylglucosaminyl | |
| | N-acetylglucosaminyl | | | transferase II, UDP- | |
| | transferase | | | GlcNAc: mannoside | |
| | | | | alpha-1-6 | |
| | | | | acetylglucosaminyl | |
| | | | | transferase, Alpha-1,6- | |
| | | | | mannosyl-glycoprotein | |
| | | | | 2-beta-N- | |
| | | | | acetylglucosaminyl | |
| | | | | transferase | |
| GnTIII | beta-1,4-mannosyl- | Golgi | 2.4.1.144 | GlcNAc transferase 3, | Mgat3 |
| | glycoprotein 4-beta-N- | | | N-acetylglucosaminyl | |
| | acetylglucosaminyl | | | transferase III | |
| | transferase | | | | |
| GnTIV | mannosyl (alpha-1,3-)- | Golgi | 2.4.1.145 | GlcNAc transferase 4, | Mgat4 |
| | glycoprotein beta-1,4- | | | N-acetylglucosaminyl | |
| | N-acetylglucosaminyl | | | transferase IV, Alpha- | |
| | transferase | | | 1,3-mannosyl- | |
| | | | | glycoprotein 4-beta-N- | |
| | | | | acetylglucosaminyl | |
| | | | | transferase, isozymes A | |
| | | | | and B | |
| GnTV | mannosyl (alpha-1,6)- | Golgi | 2.4.1.155 | GlcNAc transferase 5, | Mgat5 |
| | glycoprotein beta-1,6- | | | N-acetylglucosaminyl | |
| | N-acetyl-glucosaminyl | | | transferase V, Alpha- | |
| | transferase | | | 1,6-mannosyl- | |
| | | | | glycoprotein 6-beta-N- | |
| | | | | acetylglucosaminyl | |
| | | | | transferase | |
| GnTVI | alpha-1,6-mannosyl- | Golgi | 2.4.1.201 | GlcNAc transferase 6, | Mgat6 |
| | glycoprotein 4-beta-N- | | | N-acetylglucosaminyl | |
| | acetylglucosaminyl | | | transferase VI | |
| | transferase | | | | |
| GalT | beta-N- | Golgi | 2.4.1.38 | Gal-Transferase 8, | B4galT1 |
| | acetylglucosaminyl- | | | UDP-Gal transferase | |
| | glycopeptide beta-1,4- | | | | |
| | galactosyl transferase | | | | |
| FucT | alpha (1,6) fucosyl | Golgi | 2.4.1.68 | Fuc-transferase 8, | Fut8 |
| | transferase | | | GDP-Fuc transferase | |
| ST | beta-galactoside alpha- | Golgi | 2.4.99.1 | Sialyltransferase, CMP- | ST6gal1 |
| | 2,6-sialyl transferase | | | N-acetylneuraminate- | |
| | | | | beta-galactosamide- | |
| | | | | alpha-2,6-sialyl trans- | |
| | | | | ferase, | |
| | UDP-N- | Cytosol | 5.1.3.14 | UDP-GlcNAc-2- | NeuC |
| | acetylglucosamine 2 | | | epimerase | |
| | epimerase | | | | |
| | sialic acid synthase | Cytosol | | | NeuB |
| | CMP-NeuNAc | Cytosol | 2.7.7.43 | | Cmas |
| | synthetase | | | | NeuA |
| | N-acylneuraminate-9- | | 2.5.1.57 | | |
| | phosphate synthase | | | | |
| | N-acylneuraminate-9- | | 3.1.3.29 | | |
| | phosphatase | | | | |
| | UDP-GlcNac | Golgi | | | Slc35A3 |
| | transporter | | | | |
| | UDP-Gal-transporter | Golgi | | | Slc35A2 |
| | GDP-fucose | Golgi | | | Slc35C1 |
| | transporter | | | | |
| | CMP-sialic acid | Golgi | | | Slc35A1 |
| | transporter | | | | |
| | nucleotide | Golgi | | | |
| | diphoshatases | | | | |
| | GDP-D-mannose 4,6- | Cytosol | 4.2.1.47 | | Gmds |
| | dehydratase | | | | |
| | GDP-4-keto-6-deoxy- | Cytosol | 1.1.1.271 | GDP L-fucose | Tsta3 |
| | D-mannose-3,5- | | | synthase, FX protein | |
| | epimerase-4-reductase | | | | |
| Gal10 | UDP-glucose 4- | Cytosol | 5.1.3.2 | UDP-galactose 4- | SPBPB2 |
| | epimerase | | | epimerase, GalE | B2.12c |
| Uge1 | UDP-glucose 4- | Cytosol | 5.1.3.2 | UDP-galactose 4- | SPBC36 |
| | epimerase | | | epimerase | 5.14c |

TABLE 3
Heterologous enzymes for Golgi-based synthesis of prefered biantennary glycans

| | | | | |
|---|---|---|---|---|
| N-acetylglucosaminylation | bisecting GlcNAc | galactosylation | fucosylation | sialylation |

GlcNAcMan3-5GlcNAc2

mannosyl(alpha-1,3-)-glycoprotein
beta-1,2-N-acetylglucosaminyl
transferase (GnTI)
UDP-N-acetylglucosamine transporter

GlcNAc2Man3GlcNAc2

mannosyl(alpha-1,3-)-glycoprotein
beta-1,2-N-acetylglucosaminyl
transferase (GnTI)
UDP-N-acetylglucosamine transporter
mannosyl(alpha-1,6-)-glycoprotein
beta-1,2-N-acetylglucosaminyl
transferase (GnTII)

GlcNAc3Man3GlcNAc2-bisecting

| | |
|---|---|
| mannosyl(alpha-1,3-)-glycoprotein | beta-1,4-mannosyl- |
| beta-1,2-N-acetylglucosaminyl | glycoprotein 4-beta-N- |
| transferase (GnTI) | acetylglucosaminyl |
| UDP-N-acetylglucosamine transporter | transferase (GnTIII) |
| mannosyl(alpha-1,6-)-glycoprotein | |
| beta-1,2-N-acetylglucosaminyl | |
| transferase (GnTII) | |

Gal2GlcNAc2Man3GlcNAc2

| | |
|---|---|
| mannosyl(alpha-1,3-)-glycoprotein | beta-N-acetylglucosaminyl |
| beta-1,2-N-acetylglucosaminyl | glycopeptide beta-1,4- |
| transferase (GnTI) | galactosyl transferase |
| UDP-N-acetylglucosamine transporter | (GalT) UDP-galactose |
| mannosyl(alpha-1,6-)-glycoprotein | transporter |
| beta-1,2-N-acetylglucosaminyl | |
| transferase (GnTII) | |

Gal2GlcNAc2Man3GlcNAc2Fuc

| | | |
|---|---|---|
| mannosyl(alpha-1,3-)-glycoprotein | beta-N-acetylglucosaminyl | GDP-D-mannose 4,6- |
| beta-1,2-N-acetylglucosaminyl | glycopeptide beta-1,4- | dehydratase |
| transferase (GnTI) | galactosyl transferase | GDP-4-keto-6-deoxy- |
| UDP-N-acetylglucosamine transporter | (GalT) UDP-galactose | D-mannose-3,5- |
| mannosyl(alpha-1,6-)-glycoprotein | transporter | epimerase-4-reductase |
| beta-1,2-N-acetylglucosaminyl | | GDP-fucose transporter |
| transferase (GnTII) | | alpha (1,6) fucosyl |
| | | transferase (FucT) |

Gal2GlcNAc3Man3GlcNAc2-bisecting

| | | |
|---|---|---|
| mannosyl(alpha-1,3-)-glycoprotein | beta-1,4-mannosyl- | beta-N-acetylglucosaminyl |
| beta-1,2-N-acetylglucosaminyl | glycoprotein 4-beta-N- | glycopeptide beta-1,4- |
| transferase (GnTI) | acetylglucosaminyl | galactosyl transferase |
| UDP-N-acetylglucosamine transporter | transferase (GnTIII) | (GalT) UDP-galactose |
| mannosyl(alpha-1,6-)-glycoprotein | | transporter |
| beta-1,2-N-acetylglucosaminyl | | |
| transferase (GnTII) | | |

Gal2GlcNAc3Man3GlcNAc2Fuc-bisecting

| | | | |
|---|---|---|---|
| mannosyl(alpha-1,3-)-glycoprotein | beta-1,4-mannosyl- | beta-N-acetylglucosaminyl | GDP-D-mannose 4,6- |
| beta-1,2-N-acetylglucosaminyl | glycoprotein 4-beta-N- | glycopeptide beta-1,4- | dehydratase |
| transferase (GnTI) | acetylglucosaminyl | galactosyl transferase | GDP-4-keto-6-deoxy- |
| UDP-N-acetylglucosamine transporter | transferase (GnTIII) | (GalT) UDP-galactose | D-mannose-3,5- |
| mannosyl(alpha-1,6-)-glycoprotein | | transporter | epimerase-4-reductase |
| beta-1,2-N-acetylglucosaminyl | | | GDP-fucose transporter |
| transferase (GnTII) | | | alpha (1,6) fucosyl |
| | | | transferase (FucT) |

NeuAc2Gal2GlcNAc2Man3GlcNAc2

| | | |
|---|---|---|
| mannosyl(alpha-1,3-)-glycoprotein | beta-N-acetylglucosaminyl | beta-galactoside alpha- |
| beta-1,2-N-acetylglucosaminyl | glycopeptide beta-1,4- | 2,6-sialyl transferase |
| transferase (GnTI) | galactosyl transferase | (ST) UDP-N-acetyl- |
| UDP-N-acetylglucosamine transporter | (GalT) UDP-galactose | glucosamine |
| mannosyl(alpha-1,6-)-glycoprotein | transporter | 2-epimerase (NeuC) |
| beta-1,2-N-acetylglucosaminyl | | sialic acid synthase |
| transferase (GnTII) | | (NeuB) or: N- |
| | | acylneuraminate-9- |
| | | phosphate synthase |
| | | N-acylneuraminate-9- |
| | | phosphatase |
| | | CMP-Neu5Ac synthetase |
| | | CMP-sialic acid |
| | | transporter |

NeuAc2Gal2GlcNAc2Man3GlcNAc2Fuc

| | | | |
|---|---|---|---|
| mannosyl(alpha-1,3-)-glycoprotein | beta-N-acetylglucosaminyl | GDP-D-mannose 4,6- | beta-galactoside alpha- |
| beta-1,2-N-acetylglucosaminyl | glycopeptide beta-1,4- | dehydratase | 2,6-sialyl transferase |
| transferase (GnTI) | galactosyl transferase | GDP-4-keto-6-deoxy- | (ST) UDP-N-acetyl- |
| UDP-N-acetylglucosamine transporter | (GalT) UDP-galactose | D-mannose-3,5- | glucosamine |
| mannosyl(alpha-1,6-)-glycoprotein | transporter | epimerase-4-reductase | 2-epimerase (NeuC) |
| beta-1,2-N-acetylglucosaminyl | | GDP-fucose transporter | sialic acid synthase |
| transferase (GnTII) | | alpha (1,6) fucosyl | (NeuB) or: N- |
| | | transferase (FucT) | acylneuraminate-9- |
| | | | phosphate synthase + |
| | | | N-acylneuraminate-9- |
| | | | phosphatase |
| | | | CMP-Neu5Ac synthetase |
| | | | CMP-sialic acid |
| | | | transporter |

NeuAc2Gal2GlcNAc3Man3GlcNAc2-bisecting

| | | | |
|---|---|---|---|
| mannosyl(alpha-1,3-)-glycoprotein | beta-1,4-mannosyl- | beta-N-acetylglucosaminyl | beta-galactoside alpha- |
| beta-1,2-N-acetylglucosaminyl | glycoprotein 4-beta-N- | glycopeptide beta-1,4- | 2,6-sialyl transferase |
| transferase (GnTI) | acetylglucosaminyl | galactosyl transferase | (ST) UDP-N-acetyl- |
| UDP-N-acetylglucosamine transporter | transferase (GnTIII) | (GalT) UDP-galactose | glucosamine |
| mannosyl(alpha-1,6-)-glycoprotein | | transporter | 2-epimerase (NeuC) |
| beta-1,2-N-acetylglucosaminyl | | | sialic acid synthase |
| transferase (GnTII) | | | (NeuB) or: N- |
| | | | acylneuraminate-9- |
| | | | phosphate synthase + |
| | | | N-acylneuraminate-9- |
| | | | phosphatase |
| | | | CMP-Neu5Ac synthetase- |
| | | | CMP-sialic acid |
| | | | transporter |

NeuAc2Gal2GlcNAc3Man3GlcNAc2Fuc-bisecting

| | | | | |
|---|---|---|---|---|
| mannosyl(alpha-1,3-)-glycoprotein | beta-1,4-mannosyl- | beta-N-acetylglucosaminyl | GDP-D-mannose 4,6- | beta-galactoside alpha- |
| beta-1,2-N-acetylglucosaminyl | glycoprotein 4-beta-N- | glycopeptide beta-1,4- | dehydratase | 2,6-sialyl transferase |
| transferase (GnTI) | acetylglucosaminyl | galactosyl transferase | GDP-4-keto-6-deoxy- | (ST) UDP-N-acetyl- |
| UDP-N-acetylglucosamine transporter | transferase (GnTIII) | (GalT) UDP-galactose | D-mannose-3,5- | glucosamine |
| mannosyl(alpha-1,6-)-glycoprotein | | transporter | epimerase-4-reductase | 2-epimerase (NeuC) |
| beta-1,2-N-acetylglucosaminyl | | | GDP-fucose transporter | sialic acid synthase (NeuB |
| transferase (GnTII) | | | alpha (1,6) fucosyl | or: N-acylneuraminate-9- |
| | | | transferase (Fuel) | phosphate synthase + |
| | | | | N-acylneuraminate-9- |
| | | | | phosphatase |
| | | | | CMP-Neu5Ac synthetase |
| | | | | CMP-sialic acid |
| | | | | transporter |

TABLE 4
Heterologous enzymes for Golgi-based synthesis of preferred triantennary glycans

| | | | |
|---|---|---|---|
| N-acetylglucosaminylation | galactosylation | fucosylation | sialylation |

GlcNAc3Man3GlcNAc2

mannosyl(alpha-1,3-)-glycoprotein beta-1,2-N-
acetylglucosaminyl transferase (GnTI)
UDP-N-acetylglucosamine transporter
mannosyl(alpha-1,6-)-glycoprotein beta-1,2-N-
acetylglucosaminyl transferase (GnTII)
mannosyl(alpha-1,3-)-glycoprotein beta-1,4-N-
acetylglucosaminyl transferase(GnTIV)

Gal3GlcNAc3Man3GlcNAc2

| | |
|---|---|
| mannosyl(alpha-1,3-)-glycoprotein beta-1,2-N- | beta-N-acetylglucosaminyl glyco- |
| acetylglucosaminyl transferase (GnTI) | peptide beta-1,4-galactosyl trans- |
| UDP-N-acetylglucosamine transporter | ferase (GalT) |
| mannosyl(alpha-1,6-)-glycoprotein beta-1,2-N- | UDP-galactose transporter |
| acetylglucosaminyl transferase (GnTII) | |
| mannosyl(alpha-1,3-)-glycoprotein beta-1,4-N- | |
| acetylglucosaminyl transferase(GnTIV) | |

Gal3GlcNAc3Man3GlcNAc2Fuc

| | | |
|---|---|---|
| mannosyl(alpha-1,3-)-glycoprotein beta-1,2-N- | beta-N-acetylglucosaminyl glyco- | GDP-D-mannose 4,6- |
| acetylglucosaminyl transferase (GnTI) | peptide beta-1,4-galactosyl trans- | dehydratase GDP-4-keto- |
| UDP-N-acetylglucosamine transporter | ferase (GalT) | 6-deoxy-D-mannose-3,5- |
| mannosyl(alpha-1,6-)-glycoprotein beta-1,2-N- | UDP-galactose transporter | epimerase-4-reductase |
| acetylglucosaminyl transferase (GnTII) | | GDP-fucose transporter |
| mannosyl(alpha-1,3-)-glycoprotein beta-1,4-N- | | alpha (1,6) fucosyl |
| acetylglucosaminyl transferase(GnTIV) | | transferase (FucT) |

NeuAc3Gal3GlcNAc3Man3GlcNAc

| | | |
|---|---|---|
| mannosyl(alpha-1,3-)-glycoprotein beta-1,2-N- | beta-N-acetylglucosaminyl glyco- | 2,6-sialyl transferase (ST) |
| acetylglucosaminyl transferase (GnTI) | peptide beta-1,4-galactosyl trans- | UDP-N-acetylglucosamine 2- |
| UDP-N-acetylglucosamine transporter | ferase (GalT) | epimerase (NeuC) |
| mannosyl(alpha-1,6-)-glycoprotein beta-1,2-N- | UDP-galactose transporter | sialic acid synthase (NeuB) |
| acetylglucosaminyl transferase (GnTII) | | or: N-acylneuraminate-9- |
| mannosyl(alpha-1,3-)-glycoprotein beta-1,4-N- | | phosphate synthase + N- |
| acetylglucosaminyl transferase(GnTIV) | | acylneuraminate-9- |
| | | phosphatase CMP-Neu5Ac |
| | | synthetase CMP-sialic |
| | | acid transporter |

NeuAc3Gal3GlcNAc3Man3GlcNAcFuc

| | | | |
|---|---|---|---|
| mannosyl(alpha-1,3-)-glycoprotein beta-1,2-N- | beta-N-acetylglucosaminyl glyco- | GDP-D-mannose 4,6- | 2,6-sialyl transferase (ST) |
| acetylglucosaminyl transferase (GnTI) | peptide beta-1,4-galactosyl trans- | dehydratase GDP-4-keto- | UDP-N-acetylglucosamine 2- |
| UDP-N-acetylglucosamine transporter | ferase (GalT) | 6-deoxy-D-mannose-3,5- | epimerase (NeuC) |
| mannosyl(alpha-1,6-)-glycoprotein beta-1,2-N- | UDP-galactose transporter | epimerase-4-reductase | sialic acid synthase (NeuB) |
| acetylglucosaminyl transferase (GnTII) | | GDP-fucose transporter | or: N-acylneuraminate-9- |
| mannosyl(alpha-1,3-)-glycoprotein beta-1,4-N- | | alpha (1,6) fucosyl | phosphate synthase + N- |
| acetylglucosaminyl transferase(GnTIV) | | transferase (FucT) | acylneuraminate-9- |
| | | | phosphatase CMP-Neu5Ac |
| | | | synthetase CMP-sialic |
| | | | acid transporter |

The primary goal of this genetic engineering effort is to produce robust protein production strains that are able to perform proteins with defined, human-like glycan structures in an industrial fermentation process. The integration of multiple genes into the host (e.g., fungal) chromosome involves careful planning. The engineered strain will most likely have to be transformed with a range of different genes, and these genes will have to be transformed in a stable fashion to ensure that the desired activity is maintained throughout the fermentation process. Any combination of the enzyme activities will have to be engineered into the protein expression host cell.

With the DNA sequence information available, the skilled worker can clone DNA molecules encoding GnT activities Using standard techniques well-known to those of skill in the art, nucleic acid molecules encoding one or more GnT (or encoding catalytically active fragments thereof) may be inserted into appropriate expression vectors under the transcriptional control of promoters and other expression control sequences capable of driving transcription in a selected host cell of the invention, e.g., a fungal host such as Pichia sp., Kluyveromyces sp., Saccharomyces sp., Yarrowia sp. and Aspergillus sp., as described herein, such that one or more of these mammalian GnT enzymes may be actively expressed in a host cell of choice for production of a human-like complex glycoprotein.

The engineered strains will be stably transformed with different glycosylation related genes to ensure that the desired activity is maintained throughout the fermentation process. Any combination of the following enzyme activities will have to be engineered into the expression host. In parallel a number of host genes involved in undesired glycosylation reactions will have to be deleted.

In preferred embodiments a subset of genes, at least two genes (also named library), encoding heterologous glycosylation enzymes are transformed into the host organism, causing at first a genetically mixed population. Transformants having the desired glycosylation phenotypes are then selected from the mixed population. In a preferred embodiment, the host organism is a lower eukaryote and the host glycosylation pathway is modified by the stable expression of one or more human or animal glycosylation enzymes, yielding N-glycans similar or identical to human glycan structures. In an especially preferred embodiment, the subset of genes or “DNA library” include genetic constructs encoding fusions of glycosylation enzymes with targeting sequences for various cellular loci involved in glycosylation especially the ER, cis Golgi, medial Golgi, or trans Golgi.

In some cases the DNA library may be assembled directly from existing or wild-type genes. In a preferred embodiment however the DNA library is assembled from the fusion of two or more sub-libraries. By the in-frame ligation of the sub-libraries, it is possible to create a large number of novel genetic constructs encoding useful targeted glycosylation activities. For example, one useful sub-library includes DNA sequences encoding any combination of the enzymes and enzymatic activities set forth hereinafter.

Preferably, the enzymes are of human origin, although other eukaryotic or also procaryotic enzymes, more particular mammalian, protozoan, plant, bacterial or fungal enzymes are also useful. In a preferred embodiment, genes are truncated to give fragments encoding the catalytic domains of the enzymes. By removing endogenous targeting sequences, the enzymes may then be redirected and expressed in other cellular loci. The choice of such catalytic domains may be guided by the knowledge of the particular environment in which the catalytic domain is subsequently to be active. Another useful sub-library includes DNA sequences encoding signal peptides that result in localization of a protein to a particular locus within the ER, Golgi, or trans Golgi network. These signal sequences may be selected from the host organism as well as from other related or unrelated organisms. Membrane-bound proteins of the ER or Golgi typically may include, for example, N-terminal sequences encoding a cytosolic tail (ct), a transmembrane domain (tmd), and a stem region (sr). The ct, tmd, and sr sequences are sufficient individually or in combination to anchor proteins to the inner (lumenal) membrane of the organelle. Accordingly, a preferred embodiment of the sub-library of signal sequences includes ct, tmd, and/or sr sequences from these proteins. In some cases it is desirable to provide the sub-library with varying lengths of sr sequence. This may be accomplished by PCR using primers that bind to the 5′ end of the DNA encoding the cytosolic region and employing a series of opposing primers that bind to various parts of the stem region. Still other useful sources of signal sequences include retrieval signal peptides.

In addition to the open reading frame sequences, it is generally preferable to provide each library construct with such promoters, transcription terminators, enhancers, ribosome binding sites, and other functional sequences as may be necessary to ensure effective transcription and translation of the genes upon transformation into the host organism.

The invention thus further concerns a host cell according to the invention as described herein which is further genetically engineered or modified to express at least one preferably heterologous enzyme or catalytic domain thereof, said enzyme or catalytic domain thereof is represented in tables 3, 4, and 5 and is preferably selected from the group of Golgi-based heterologous enzymes consisting of:
mannosyl(alpha-1,3-)-glycoprotein beta-1,2-N-acetylglucosaminyl transferase (GnTI);
mannosyl(alpha-1,6-)-glycoprotein beta-1,2-N-acetylglucosaminyl transferase (GnTII);
beta-1,4-mannosyl-glycoprotein 4-beta-N-acetylglucosaminyl transferase or N-acetylglucosaminyl transferase III (GnTIII);
mannosyl (alpha-1,3-)-glycoprotein beta-1,4-N-acetylglucosaminyl transferase or N-acetylglucosaminyl transferase IV (GnTIV);
mannosyl (alpha-1,6-)-glycoprotein beta-1,6-N-acetyl-glucosaminyl transferase or N-acetylglucosaminyl transferase V (GnTV); alpha-1,6-mannosyl-glycoprotein 4-beta-N-acetylglucosaminyl transferase or N-acetylglucosaminyl transferase VI (GnTVI);
beta-N-acetylglucosaminyl glycopeptide beta-1,4-galactosyl transferase or galactosyl transferase (GalT);
alpha (1,6) fucosyl transferase or fucosyl transferase (FucT); beta-galactoside alpha-2,6-sialyl transferase or sialyl transferase (ST)

These enzyme activities may be further supported by the activity of one or more of the following: UDP-GlcNAc transferase; UDP-GlcNac transporter; UDP-galactosyl transferase, UDP-galactose transporter; GDP-fucosyl transferase; GDP-fucose transporter; CMP-sialyl transferase CMP-sialic acid transporter; and nucleotide diphoshatases.

In another variant, these enzyme activities are further supported by the activity of one or more of the following: UDP-GlcNAc transferase; UDP-GlcNac transporter; UDP-galactosyl transferase, UDP-galactose transporter; GDP-fucosyl transferase; GDP-fucose transporter; CMP-sialyl transferase CMP-sialic acid transporter; nucleotide diphoshatases, GDP-D-mannose 4,6-dehydratase, and GDP-4-keto-deoxy-D-mannose-3,5-epimerase-4-reductase.

In another variant, these enzyme activities are further supported by the activity of one or more of the following: UDP-GlcNAc transferase; UDP-GlcNac transporter; UDP-galactosyl transferase, UDP-galactose transporter; GDP-fucosyl transferase; GDP-fucose transporter; CMP-sialyl transferase CMP-sialic acid transporter; nucleotide diphoshatases, GDP-D-mannose 4,6-dehydratase, GDP-4-keto-deoxy-D-mannose-3,5-epimerase-4-reductase, UDP-glucose 4-epimerase, and UDP-galactose 4-epimerase.

It goes without saying that said at least one enzyme or catalytic domain described herein preferably comprises at least a localization sequence for an intracellular membrane or organelle. In the preferred embodiments the intracellular membrane or organelle is the Golgi.

In preferred variants thereof, N-acetylglucosaminyl transferase V (GnTV) and/or N-acetylglucosaminyl transferase VI (GnTVI) are not present or are lacking in the modified cell. In these variants the modifications catalyzed by one or both of these two enzyme activities are not required or excluded from the Golgi-based modification.

Embodiments for the Synthesis of GlcNAcMan3-5GlcNAc2 Structures

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript.

In a particular embodiment the cell expresses f the following genes: mgat1 and slc35A3 and/or homologues thereof.

This cell is particularly capable of producing N-glycan with GlcNAcMan3-5GlcNAc2 structures. The invention thus also concerns a host cell or a plurality thereof that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a GlcNAc2Man3GlcNAc2 Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript; and
mannosyl (alpha-1,6-)-glycoprotein beta-1,2-N-acetylglucosaminyl transferase (GnTII), in particular a Mgat2-type transcript.

In a more preferred embodiment, this cell comprises at least all of or exclusively these Golgi processing associated enzyme activities.

In a particular embodiment the cell expresses two or more of one of the following genes: mgat1, mgat2, and slc35A3 and/or homologues thereof.

This cell is particularly capable of producing N-glycan with GlcNAc2Man3GlcNAc2 structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a GlcNAc3Man3GlcNAc2-Bisecting

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript; and
beta-1,4-mannosyl-glycoprotein 4-beta-N-acetylglucosaminyl transferase (GnTIII), in particular a Mgat3-type transcript.

In a more preferred embodiment, this cell comprises at least all of or exclusively these Golgi processing associated enzyme activities.

In a preferred variant of this embodiment the cell expresses one or more of one of the following genes: mgat1, mgat2, mgat3, and slc35A3 and/or homologues thereof.

This cell is particularly capable of producing N-glycan with GlcNAc3Man3GlcNAc2-bisecting structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a Gal2GlcNAc2Man3GlcNAc2 Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript; and
UDP-galactose transporter type activity, in particular a Slc35A2-type transcript.

In a most preferred embodiment, this cell comprises at least all of or exclusively these Golgi processing associated enzyme activities.

In a preferred variant of this embodiment the cell expresses one or more of one of the following genes: mgat1, mgat2, mgat3, b4galt1, slc35a2 and slc35a3 and/or homologues thereof.

This cell is particularly capable of producing N-glycan with Gal2GlcNAc2Man3GlcNAc2 structure. The invention thus also concerns a host cell or a plurality thereof, which is specifically designed to produce glycoproteins with this glycan structure. The invention thus also concerns a, preferably isolated, glyco-protein having this structure, which is preferably producible or actually produced by this cell. The invention also provides a method or process for making that glycoprotein by using this cell.

Embodiments for the Synthesis of a Gal2GlcNAc2Man3GlcNAc2Fuc Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl (alpha-1,6-)-glycoprotein beta-1,2-N-acetylglucosaminyl transferase (GnTII), in particular a Mgat2-type transcript; and
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript; and
alpha (1,6) fucosyl transferase (FucT) type activity, in particular a Fut8-type transcript.

In a most preferred embodiment, this cell comprises at least all of or exclusively these Golgi processing associated enzyme activities.

In a preferred variant of this embodiment the cell expresses one or more of one of the following genes: mgat1, mgat2, slc35a3, mgat3, b4galt1, slc35a2, gmds, tsta3, slc35c1 and fut8; and/or homologues thereof.

This cell is particularly capable of producing N-glycan with Gal2GlcNAc2Man3GlcNAc2Fuc structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a Gal2GlcNAc3Man3GlcNAc2-Bisecting Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript.
beta-N-acetylglucosaminyl glycopeptide beta-1,4-galactosyl transferase (GalT), in particular a B4galt1-type transcript; and
UDP-galactose transporter type activity, in particular a Slc35A2-type transcript.

In a most preferred embodiment, this cell comprises at least all of or exclusively these Golgi processing associated enzyme activities.

In a preferred variant of this embodiment the cell expresses one or more of one of the following genes: mgat1, mgat2, mgat3, slc35a3, b4galt1, and slc35a2; and/or homologues thereof.

This cell is particularly capable of producing N-glycan with Gal2GlcNAc3Man3GlcNAc2-bisecting structure. The invention thus also concerns a host cell or a plurality thereof, that is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a Gal2GlcNAc3Man3GlcNAc2Fuc-Bisecting Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript.
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript; and
alpha (1,6) fucosyl transferase (FucT) type activity, in particular a Fut8-type transcript.

In a most preferred embodiment, this cell comprises at least all of or exclusively these Golgi processing associated enzyme activities.

In a preferred variant of this embodiment the cell expresses one or more of one of the following genes: mgat1, mgat2, mgat3, slc35a3, b4galt1, slc35a2, gmds, tsta3, slc35c1 and fut8 and/or homologues thereof.

This cell is particularly capable of producing N-glycan with Gal2GlcNAc3Man3GlcNAc2Fuc-bisecting structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a NeuAc2Gal2GlcNAc2Man3GlcNAc2 Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In a preferred variant of these embodiments the cell expresses one or more of one of the following genes: mgat1, mgat2, slc35a3, b4galt1, slc35a2, st6gal1, neuC, neuB, slc35a1, and neuC/cmas; and/or homologues thereof.

This cell is particularly capable of producing N-glycan with NeuAc2Gal2GlcNAc2Man3GlcNAc2 structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a NeuAc2Gal2GlcNAc3Man3GlcNAc2-bisecting structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In a preferred variant of these embodiments the cell expresses one or more of one of the following genes: mgat1, mgat2, slc35a3, mgat3, b4galt1, slc35a2, st6gal1, neuC, neuB, slc35a1, and neuC/cmas; and/or homologues thereof.

This cell is particularly capable of producing N-glycan with NeuAc2Gal2GlcNAc3Man3GlcNAc2-bisecting structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a NeuAc2Gal2GlcNAc2Man3GlcNAc2Fuc Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In a preferred variant of these embodiments the cell expresses one or more of one of the following genes: mgat1, mgat2, slc35a3, b4galt1, slc35a2, gmds, tsta3, slc35c1, fut8, st6gal1, neuC, neuB, slc35a1, and neuC/cmas; and/or homologues thereof.

This cell is particularly capable of producing N-glycan with NeuAc2Gal2GlcNAc2Man3GlcNAc2Fuc structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a NeuAc2Gal2GlcNAc3Man3GlcNAc2Fuc-Bisecting Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
beta-1,4-mannosyl-glycoprotein 4-beta-N-acetylglucosaminyl transferase (GnTIII), in particular a Mgat3-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In a preferred variant of these embodiments the cell expresses one or more of one of the following genes: mgat1, mgat2, slc35a3, b4galt1, mgat3, slc35a2, gmds, tsta3, slc35c1, fut8, st6gal1, neuC, neuB, slc35a1, and neuC/cmas; and/or homologues thereof.

This cell is particularly capable of producing N-glycan with NeuAc2Gal2GlcNAc3Man3GlcNAc2Fuc-bisecting structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a GlcNAc3Man3GlcNAc2 Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript; and
mannosyl(alpha-1,3-)-glycoprotein beta-1,4-N-acetylglucosaminyl transferase (GnTIV), in particular a Mgat-4-type transcript.

In a most preferred embodiment, this cell comprises at least all of or exclusively these Golgi processing associated enzyme activities.

In a preferred variant of this embodiment the cell expresses one or more of one of the following genes: mgat1, mgat2, mgat4, and slc35A3; and/or homologues thereof.

This cell is particularly capable of producing N-glycan with GlcNAc3Man3GlcNAc2 structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a Gal3GlcNAc3Man3GlcNAc2 Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase (GnTIV), in particular a Mgat-4-type transcript;
beta-N-acetylglucosaminyl glycopeptide beta-1,4-galactosyl transferase (GalT), in particular a B4galt1-type transcript; and
UDP-galactose transporter type activity, in particular a Slc35A2-type transcript.

In a most preferred embodiment, this cell comprises at least all of or exclusively these Golgi processing associated enzyme activities.

In a preferred variant of this embodiment the cell expresses one or more of one of the following genes: mgat1, mgat2, maga4, slc35a3, b4galt1 and slc35a2; and/or homologues thereof.

This cell is particularly capable of producing N-glycan with Gal3-GlcNAc3Man3GlcNAc2 structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a Gal3GlcNAc3Man3GlcNAc2Fuc Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl (alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase (GnTIV), in particular a Mgat-4-type transcript;
beta-N-acetylglucosaminyl glycopeptide beta-1,4-galactosyl transferase (GalT), in particular a B4galt1-type transcript; and
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript; and
alpha (1,6) fucosyl transferase (FucT) type activity, in particular a Fut8-type transcript.

In a most preferred embodiment, this cell comprises at least all of or exclusively these Golgi processing associated enzyme activities.

In a preferred variant of this embodiment the cell expresses one or more of one of the following genes: mgat1, mgat2, maga4, slc35a3, b4galt1, slc35a2, gmds, tsta3, slc35c1 and fut8; and/or homologues thereof.

This cell is particularly capable of producing N-glycan with Gal3-GlcNAc3Man3GlcNAc2Fuc structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a NeuAc3Gal3GlcNAc3Man3GlcNAc2 Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase (GnTIV), in particular a Mgat-4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the modified host cell exhibits, preferably heterologous, enzyme activity for Golgi-based processing that is selected from:
mannosyl(alpha-1,3-)-glycoprotein beta-1,2-N-acetylglucosaminyl transferase (GnTI) type activity, in particular a Mgat1-type transcript;
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase (GnTIV), in particular a Mgat-4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In a preferred variant of these embodiments the cell expresses one or more of one of the following genes: mgat1, mgat2, slc35a3, b4galt1, mgat4, slc35a2, st6gal1, neuC, neuB, slc35a1, and neuC/cmas; and/or homologues thereof.

This cell is particularly capable of producing N-glycan with NeuAc3Gal3GlcNAc3Man3GlcNAc2 structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

Embodiments for the Synthesis of a NeuAc3Gal3GlcNAc3Man3GlcNAc2Fuc Structure

In a preferred embodiment the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase (GnTIV), in particular a Mgat-4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
sialic acid synthase (NeuB), in particular a NeuB-type transcript;
CMP-Neu5Ac synthetase, in particular a NeuA/Cmas-type transcript; and
CMP-sialic acid transporter, in particular a Slc35A1-type transcript.

In a most preferred embodiment, this cell comprises at least all of or exclusively these Golgi processing associated enzyme activities.

In an alternative variant thereof, the modified host cell exhibits N-acylneuraminate-9-phosphate synthase and N-acylneuraminate-9-phosphatase activity instead of sialic acid synthase activity, more particular the modified host cell not only exhibits, preferably heterologous, enzyme activity for Golgi-based processing that is selected from GnTI type activity, in particular a Mgat1-type transcript, but also comprise a, preferably heterologous, enzyme activity that is selected from:
UDP-N-acetylglucosamine transporter type activity, in particular a Slc35A3-type transcript;
mannosyl(alpha-1,6-)-glycoprotein beta-1,2-N-acetylglucosaminyl transferase (GnTII), in particular a Mgat2-type transcript;
mannosyl(alpha-1,3-)-glycoprotein beta-1,4-N-acetylglucosaminyl transferase (GnTIV), in particular a Mgat-4-type transcript;
beta-N-acetylglucosaminyl glycopeptide beta-1,4-galactosyl transferase (GalT), in particular a B4galt1-type transcript;
UDP-galactose transporter type activity, in particular a Slc35A2-type transcript;
GDP-D-mannose 4,6-dehydratase type activity, in particular a Gmds-type transcript;
GDP-4-keto-6-deoxy-D-mannose-3,5-epimerase-4-reductase type activity, in particular a Tsta3-type transcript;
GDP-fucose transporter type activity, in particular a Slc35C1-type transcript;
alpha (1,6) fucosyl transferase (FucT) type activity, in particular a Fut8-type transcript;
beta-galactoside alpha-2,6-sialyl transferase (ST), in particular a ST6gal1-type transcript;
UDP-N-acetylglucosamine 2-epimerase (NeuC), in particular a NeuC-type transcript;
N-acylneuraminate-9-phosphate synthase;
N-acylneuraminate-9-phosphatase;
CMP-Neu5Ac synthetase, in particular a Slc35A1-type transcript; and
CMP-sialic acid transporter, in particular a NeuA/Cmas-type transcript.

In a most preferred embodiment, this cell comprises at least all of or exclusively these Golgi processing associated enzyme activities.

In a preferred variant of these embodiments the cell expresses one or more of one of the following genes: mgat1, mgat2, slc35a3, b4galt1, mgat4, slc35a2, gmds, tsta3, slc35c1, fut8, st6gal1, neuC, neuB, slc35a1, and neuC/cmas; and/or homologues thereof.

This cell is particularly capable of producing N-glycan with NeuAc3Gal2GlcNAc3Man3GlcNAc2Fuc structure. The invention thus also concerns a host cell or a plurality thereof, which is specifically designed to produce glycoproteins with this glycan structure. The invention thus also concerns a, preferably isolated, glycoprotein having this structure, which is preferably producible or actually produced by this cell. The invention also provides a method or process for making that glycoprotein by using this cell.

The invention also provides a method or process for making a glycoprotein by using any one of the host cells according to the invention. Without wishing to be bound to the theory, a cell according to the invention is capable of producing high amounts of a N-Glycan with Man3GlcNac2 structure on said glycoprotein. The glycoprotein may be a homologous or a heterologous protein. Accordingly, any one of the host cells as outlined above preferably comprise at least one nucleic acid encoding a heterologous glycoprotein. Homologous proteins primarily refers to proteins from the host cell itself, whereas proteins encoded by “foreign”, cloned genes are heterologous proteins of the host cell. More particular, any nucleic acid encoding a heterologous protein according to the invention can be codon-optimized for expression in the host cell of interest. For example, a nucleic acid encoding a murine GnTI activity of (Mus musculus) can be codon-optimized for expression in a yeast cell such as Saccharomyces cerevisiae.

The host cell according to the invention is capable of producing complex N-linked oligosaccharides and hybrid oligosaccharides. Branched complex N-glycans have been implicated in the physiological activity of therapeutic proteins, such as human erythropoietin (hEPO). Human EPO having bi-antennary structures has been shown to have a low activity, whereas hEPO having tetra-antennary structures resulted in slower clearance from the bloodstream and thus in higher activity (Misaizu T et al. (1995) Blood 86(11):4097-104).

A glycan structure means an oligosaccharide bound to a protein core. High mannose structures contain more than 5 mannoses whereas glycan structures consisting primarily of mannose but only to an extend of 5 or less mannose moieties are low mannose glycan structures, i.e. Man3-5GlcNac2. More particular, as used herein, the term “glycan” or “glycoprotein” refers to an N-linked oligosaccharide, e.g., one that is attached by an asparagine-N-acetylglucosamine linkage to an asparagine residue of a polypeptide. N-glycans have a common pentasaccharide core of Man3GlcNAc2 (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., fucose and sialic acid) that are added to the Man3GlcNAc2 (“Man3”) core structure. N-glycans are classified according to their branched constituents (e.g., high mannose, complex or hybrid). A glycoform represents a glycosylated protein which carries a specific N-glycan. Therefore, glycoforms represent glycosylated proteins carrying different N-glycans. A “high mannose” type N-glycan has five or more mannose residues.

Common to all classes of N-glycans is the core structure Man3GlcNac2. The core structure is followed by an extension sequence on each branch, terminated by a cell-type specific hexose. Three general types of N-glycan structures could be defined: (1) High-mannose glycans, which contain mainly mannoses within their extension sequences and also as terminating moiety. (2) Complex glycans in contrast are composed of different hexoses and amino sugars. In humans they often contain N-acetylnauraminic acid as terminal sugar. And (3) hybrid glycans contain both, poly-mannosylic and complex type extension sequences within one “antenna” or molecule branch.

A “complex” type N-glycan typically has at least one GlcNAc attached to the 1,3 mannose arm and at least one GlcNAc attached to the 1,6 mannose arm of a “tri-mannose” core. The “trimannose core” is the pentasaccharide core having a Man3 structure. Complex N-glycans may also have galactose (“Gal”) residues that are optionally modified with sialic acid or derivatives (“NeuAc”, where “Neu” refers to neuraminic acid and “Ac” refers to acetyl). Complex N-glycans may also have intrachain substitutions comprising “bisecting” GlcNAc and core fucose (“Fuc”). A “hybrid” N-glycan has at least one GlcNAc on the terminal of the 1,3 mannose arm of the trimannose core and zero or more mannoses on the 1,6 mannose arm of the trimannose core.

A further aspect of the invention is a process for making a glycoprotein with a low mannose glycan structure or a glycoprotein-composition comprising one or more glycoproteins having low mannose glycan structure.

In a preferred embodiment the protein is an heterologous protein. In a preferred variant thereof the heterologous protein is a recombinant protein. A preferred embodiment of the invention is a composition that is comprising an heterologous and/or recombinant glycoprotein that is produced or producible by the cell of the invention, wherein the composition comprises a high yield of glycoprotein having a glycan structure of Man3GlcNAc2

“Recombinant protein”, “heterologous protein” and “heterologous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

In a preferred variant there is provided a process for making a glycoprotein with a Man3GlcNAc2 glycan structure or a glycoprotein-composition comprising at least one glycoprotein with a Man3GlcNAc2 glycan structure. In another preferred variant there is also provided a process for making a human-like glycoprotein with a Man4GlcNAc2 glycan structure or a glycoprotein-composition comprising at least one glycoprotein with a Man4GlcNAc2 glycan structure. In another preferred variant there is also provided a process for making a human-like glycoprotein with a Man5GlcNAc2 glycan structure or a glycoprotein-composition comprising at least one glycoprotein with a Man5GlcNAc2 glycan structure.

The process comprises at least the following step: Provision of a mutant cell according to the invention, which further transformed to be capable of producing a recombinant protein of interest, e.g. EPO or IgG. The cell is cultured in a preferably liquid culture medium and preferably under conditions that allow or most preferably support the production of said glycoprotein or glycoprotein composition in the cell. If necessary, required said glycoprotein or glycoprotein composition may be isolated from said cell and/or said culture medium. The isolation is preferably performed using methods and means known in the art.

The invention also provides new glycoproteins and compositions thereof, which are producible or are produced by the cells or methods according to the invention. Such compositions are further characterized in comprising glycan core structures selected from Man5GlcNAc2, Man4GlcNAc2, and Man3GlcNAc2, preferably a Man3GlcNAc2 structure. The invention may also provide compositions characterized in comprising glycan structures selected from Man4GlcNAc2 and Man5GlcNAc2, which may be produced due to further mannosylation of said Man3GlcNAc2 core in the Golgi.

In preferred embodiments one or more said glycan structure is present in the composition in an amount of at least 40% or more, more preferred at least 50% or more, even more preferred 60% or more, even more preferred 70% or more, even more preferred 80% or more, even more preferred 90% or more, even more preferred 95% or more, most preferred to 99% or 100%. It goes without saying that other substances and by-products that are common to such protein compositions are excluded from that calculation. In a most preferred embodiment basically all glycan structures produced by the cell exhibit a Man3GlcNAc2 structure. In another preferred embodiment basically all glycoforms produced by the cell exhibit a Man4GlcNAc2 and/or a Man5GlcNAc2 structure.

As the result of the Golgi-modification, as described hereinabove in more detail, a glycoprotein carrying complexes as well as hybrid N-glycans are obtainable. The glycoproteins comprise glycan structures selected from, but not limited to:
Man3GlcNAc2
Man4GlcNAc2
Man5GlcNAc2,
GlcNAcMan3GlcNAc2,
GlcNAcMan4GlcNAc2,
GlcNAcMan5GlcNAc2,
GlcNAc2Man3GlcNAc2,
GlcNAc3Man3GlcNAc2-bisecting
Gal1GlcNAc2Man3GlcNAc2,
Gal1GlcNAc2Man3GlcNAc2Fuc,
Gal1GlcNAc3Man3GlcNAc2-bisecting,
Gal1GlcNAc3Man3GlcNAc2Fuc-bisecting,
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc1Gal2GlcNAc2Man3GlcNAc2,
NeuAc1Gal2GlcNAc2Man3GlcNAc2Fuc,
NeuAc1Gal2GlcNAc3Man3GlcNAc2-bisecting,
NeuAc1Gal2GlcNAc3Man3GlcNAc2Fuc-bisecting,
NeuAc2Gal2GlcNAc2Man3GlcNAc2,
NeuAc2Gal2GlcNAc2Man3GlcNAc2Fuc,
NeuAc2Gal2GlcNAc3Man3GlcNAc2-bisecting,
NeuAc2Gal2GlcNAc3Man3GlcNAc2Fuc-bisecting,
GlcNAc3Man3GlcNAc2,
Gal1GlcNAc3Man3GlcNAc2,
Gal1GlcNAc3Man3GlcNAc2Fuc,
Gal2GlcNAc3Man3GlcNAc2,
Gal2GlcNAc3Man3GlcNAc2Fuc,
Gal3GlcNAc3Man3GlcNAc2,
Gal3GlcNAc3Man3GlcNAc2Fuc,
NeuAc1Gal3GlcNAc3Man3GlcNAc2,
NeuAc1Gal3GlcNAc3Man3GlcNAc2Fuc,
NeuAc2Gal3GlcNAc3Man3GlcNAc2,
NeuAc2Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

Particular embodiments include:
GlcNAcMan3GlcNAc2,
GlcNAcMan4GlcNAc2,
GlcNAcMan5GlcNAc2,
GlcNAc2Man3GlcNAc2,
GlcNAc3Man3GlcNAc2-bisecting
Gal2GlcNAc2Man3GlcNAc2,
Gal2GlcNAc2Man3GlcNAc2Fuc,
Gal2GlcNAc3Man3GlcNAc2-bisecting,
Gal2GlcNAc3Man3GlcNAc2Fuc-bisecting,
NeuAc2Gal2GlcNAc2Man3GlcNAc2,
NeuAc2Gal2GlcNAc2Man3GlcNAc2Fuc,
NeuAc2Gal2GlcNAc3Man3GlcNAc2-bisecting,
NeuAc2Gal2GlcNAc3Man3GlcNAc2Fuc-bisecting,
GlcNAc3Man3GlcNAc2,
Gal3GlcNAc3Man3GlcNAc2,
Gal3GlcNAc3Man3GlcNAc2Fuc,
NeuAc3Gal3GlcNAc3Man3GlcNAc2, and
NeuAc3Gal3GlcNAc3Man3GlcNAc2Fuc.

In preferred embodiments one or more of the above-identified glycan structures is present in the glycoprotein or glycoprotein composition in an amount of at least about 40% or more, more preferred at least about 50% or more, even more preferred about 60% or more, even more preferred about 70% or more, even more preferred 80% or more, even more preferred about 90% or more, even more preferred about 95% or more, and most preferred 99% to all glycoproteins. It goes without saying that other substances and by-products that are common to such protein compositions are excluded from that calculation. In a most preferred embodiment basically all glycoproteins that are produced by the host cell of the invention exhibit one or more of the above-identified glycan structures.

In some embodiments, the N-glycosylation form of the glycoprotein according to the invention can be homogenous or substantially homogenous. In particular, the fraction of one particular glycan structure in the glycoprotein is at least about 20% or more, about 30% or more, about 40% or more, more preferred at least about 50% or more, even more preferred about 60% or more, even more preferred about 70% or more, even more preferred 80% or more, even more preferred about 90% or more, even more preferred about 95% or more, and most preferred 99% to all glycoproteins.

Preferred embodiments of the invention are novel glycoprotein compositions that are produced or are producible by the host cells exhibiting at two or more different glycoproteins of the above-identified glycan structures. Without wishing to be bound to the theory, in a preferred embodiment a particular host cell of the invention is capable of producing two or more different at the same time, which results in “mixtures” of glycoproteins of different structure. This also refers to intermediate forms of glycosylation. It must be noted that in most preferred variants of the invention the host cell provides to an essential extend, mainly or even purely (more than 90%, preferably more than 95%, most preferred 99% or more), one particular glycan structure.

In another preferred embodiment, two or more different host cells of the invention that preferably are co-cultivated to produce two or more different N-glycan structures, which results in “mixtures” of glycoproteins of different structure.

Instrumentation suitable for N-glycan analysis includes, e.g., the ABI PRISM® 377 DNA sequencer (Applied Biosystems). Data analysis can be performed using, e.g., GENESCAN® 3.1 software (Applied Biosystems). Additional methods of N-glycan analysis include, e.g., mass spectrometry (e.g., MALDI-TOF-MS), high-pressure liquid chromatography (HPLC) on normal phase, reversed phase and ion exchange chromatography (e.g., with pulsed amperometric detection when glycans are not labeled and with UV absorbance or fluorescence if glycans are appropriately labeled).

A preferred embodiment is a recombinant immunoglobulin such as an IgG, producible by the cell of the invention, comprising N-glycan of Gal2GlcNAc2Man3GlcNAc2 structure.

Another preferred embodiment is a recombinant human Erythropoetin (rhuEPO), producible by the cell of the invention, comprising three N-glycans of NeuAc3Gal3GlcNAc3Man3GlcNAc2Fuc structure.

In preferred embodiments the glycoproteins or glycoprotein compositions can, but need not, be isolated from the host cells. In preferred embodiments the glycoproteins or glycoprotein compositions can, but need not, be further purified from the host cells. As used herein, the term “isolated” refers to a molecule, or a fragment thereof, which has been separated or purified from components, for example, proteins or other naturally-occurring biological or organic molecules, which naturally accompany it. Typically, an isolated glycoprotein or glycoprotein composition of the invention constitutes at least 60%, by weight, of the total molecules of the same type in a preparation, e.g., 60% of the total molecules of the same type in a sample. For example, an isolated glycoprotein constitutes at least 60%, by weight, of the total protein in a preparation or sample. In some embodiments, an isolated glycoprotein in the preparation consists of at least 75%, at least 90%, or at least 99%, by weight, of the total molecules of the same type in a preparation.

The genetically engineered host cells can be used in methods to produce novel glycoprotein or compositions thereof that are therapeutically active.

Preferred glycoproteins or glycoprotein compositions that are produced or are producible by the host cells according the above identified preferred embodiments include, but are not limited to, blood factors, anticoagulants, thrombolytics, antibodies, antigen-binding fragments thereof, hormones, growth factors, stimulating factors, chemokines, and cytokines, more particularly, regulatory proteins of the TFN-family, erythropoietin (EPO), gonadotropins, immunoglobulins, granulocyte-macrophage colony-stimulating factors, interferons, and enzymes. Most preferred glycoproteins or glycoprotein compositions are selected from: erythropoietin (EPO), interferon-[alpha], interferon-[beta], interferon-[gamma], interferon-[omega], and granulocyte-CSF, factor VIII, factor IX, human protein C, soluble IgE receptor [alpha]-chain, immunoglobuline-G (IgG), Fab of IgG, IgM, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, glucocerebrosidase, galactocerebrosidase, alpha-L-iduronidase, beta-D-galactosidase, beta-glucosidase, beta-hexosaminidase, beta-D-mannosidase, alpha-L-fucosidase, arylsulfatase B, arylsulfatase A, alpha-N-acteylgalactosaminidase, aspartylglucosaminidase, iduronate-2-sulfatase, alpha-glucosaminide-N-acetyltransferase, beta-D-glucoronidase, hyaluronidase, alpha-L-mannosidase, alpha-neuraminidase, phosphotransferase, acid lipase, acid ceramidase, sphinogmyelinase, thioesterase, cathepsin K, and lipoprotein lipase.

Another embodiment of the invention is a recombinant therapeutically active protein or a plurality of such proteins which is comprising one or more of the above-identified glycoproteins, in particular glycoproteins having an above-identified low-mannose glycan structure. The therapeutically active protein is preferably producible by the cell according to the present invention.

A preferred embodiment thereof is an immunoglobulin or a plurality of immunoglobulins. Another preferred embodiment thereof is an antibody or antibody-composition comprising one or more of the above-identified immunoglobulins. The term “immunoglobulin” refers to any molecule that has an amino acid sequence by virtue of which it specifically interacts with an antigen and wherein any chains of the molecule contain a functionally operating region of an antibody variable region including, without limitation, any naturally occurring or recombinant form of such a molecule such as chimeric or humanized antibodies. As used herein, “immunoglobulin” means a protein which consists of one or more polypeptides essentially encoded by an immunoglobulin gene. The immunoglobulin of the present invention preferably encompasses active fragments, preferably fragments comprising one or more glycosylation site. The active fragments mean fragments of antibody having an antigen-antibody reaction activity, and include F(ab′)2, Fab′, Fab, Fv, and recombinant Fv.

Yet another preferred embodiment is a pharmaceutical composition which is comprising one or more of the following: one or more of the above-identified glycoprotein or glycoprotein-composition according the invention, one or more of the above-identified recombinant therapeutic protein according the invention, one or more of the above-identified immunoglobulin according the invention, and one or more of the above-identified antibody according the invention. If necessary or applicable, the composition further comprises at least one pharmaceutically acceptable carrier or adjuvant.

The glycoproteins of the invention can be formulated in pharmaceutical compositions. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal or patch routes.

Pharmaceutical compositions for oral administration may be in tablet, capsule, powder or liquid form. A tablet may include a solid carrier such as gelatine or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles. Preservatives, stabilizers, buffers, antioxidants and/or other additives may be included, as required.

Whether it is a polypeptide, peptide, or nucleic acid molecule, other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners.

In another aspect, the invention provides a method of treating a disorder treatable by administration of one or more of the above-identified glycoproteins or compositions thereof, the method comprising the step(s) of: administering to a subject the glycoprotein or composition as described above, wherein the subject is suffering from, or is suspected to, a disease treatable by administration of that glycoprotein or composition. In a preferred embodiment, the method also includes the steps of (a) providing a subject and/or (b) determining whether the subject is suffering from a disease treatable by administration of said glycoprotein or composition. The subject can be mammal such as a human. The disorder can be, for example, a cancer, an immunological disorder, an inflammatory condition or a metabolic disorder.

According to the invention, there is also provided a kit or kit-of-parts for producing a glycoprotein, the kit is comprising at least: one or more host cells according to the invention, that are capable of producing the recombinant protein, and preferably a culture medium for culturing the cell so as to produce the recombinant protein.

### DESCRIPTION OF THE DRAWINGS

FIG. 1 depicts a schematic representation of biosynthetic lipid-linked oligosaccharide (LLO) pathway in yeast. LLO synthesis is initiated at the outer membrane of the ER, upon generation of Man5GlcNAc2 (M5) structure, the LLO is flipped into the ER lumen and the LLO synthesis is completed. The oligosaccharide is transferred to the protein by the OT (OST).

FIG. 2 depicts MALDI-TOF MS spectra of 2-AB-labeled N-glycans isolated from cell wall proteins from wild type cells (FIG. 2A), from Δalg3Δalg11 yeast mutant strain (FIG. 2B) and from Δalg11Δalg3Δmnn1 yeast mutant strain (FIG. 2C). The individual N-glycan peaks are annotated above the respective peaks, being Man3GlcNAc2 (M3) to Man13GlcNAc2 (M13). In addition to Mannose each indicated structure contains two additional GlcNAc (Gn) residues. These additional GlcNAc residues are the both proximal GlcNAcs of the eukaryotic N-glycan core structure. The peaks at m/z 1053 represent M3, at m/z 1215 M4, at m/z 1377 M5 and at m/z 1539 M6. The ER synthesized Man3GlcNAc2 LLO structure in Δalg3Δalg11 and Δalg11Δalg3Δmnn1 strain is further extended in the Golgi compartment to Man4GlcNAc2, Man5GlcNAc2 and very small amounts of Man6GlcNAc2. Deletion of the gene encoding Golgi localized MNN1 gene partially abolished processing of ER synthesized Man3GlcNAc2 structure in the Golgi as revealed by the strong reduction of Man5GlcNAc2 peak in Δalg11Δalg36 nm n1 strain.

FIG. 3 depicts MALDI-TOF MS spectra of permethylated N-glycans isolated from cell wall proteins from Δalg11Δalg3Δmnn1 strain carrying plasmid encoding Kre2-GnTI fusion under the control of the galactose inducible GALs promoter. Cells were induced for 17 hours with 2% galactose and grown at 26° C. (FIG. 3B) and 30° C. (FIG. 3C). The non-induced control culture (FIG. 3A) was grown at 26° C. Induction of Kre2-GnTI yields additional peaks at m/z of 1417 and m/z of 1621 representing GlcNac1Man3 (GnM3) and GlcNAc1Man4 (GnM4) carrying additional GlcNAc residue.

FIG. 4 depicts MALDI-TOF MS spectra of permethylated N-glycans isolated from cell wall proteins from Δalg11Δalg3 strain carrying plasmid encoding Kre2-GnTI fusion under the control of the galactose inducible GALs promoter. Cells were induced for 24 hours with 2% galactose and grown at 26° C. (FIG. 4B). The non-induced control culture (FIG. 4A) was grown at 26° C. Induction of hGnTI yields additional peaks at m/z of 1417 and m/z of 1621 representing GlcNac1Man3 (GnM3) and GlcNAc1Man4 (GnM4) carrying additional GlcNAc residue.

FIG. 5 depicts MALDI-TOF MS spectra of permethylated N-glycans isolated from cell wall proteins from Δalg11Δalg3 strain carrying plasmid encoding Kre2-GnTI fusion and Mnn2-GnTII fusion under the control of the galactose inducible GAL1-10 promoter. The non-induced control cells were harvested before induction (FIG. 5A). Cells were induced for 36 hours with 2% galactose. Induction of Kre2-GnTI fusion and Mnn2-GnTII fusion yields additional peaks at m/z of 1417, at m/z 1621, and at m/z of 1661 representing the hybrid structures GlcNac1Man3 (GnM3) and GlcNAc1Man4 (GnM4) and the complex N-glycan structure GlcNAc2Man3 (Gn2M3) (FIG. 5B).

FIG. 6 depicts MALDI-TOF MS spectra of permethylated N-glycans isolated from cell wall proteins from Δalg11Δalg3 (FIG. 6A) and Δalg11Δalg3Δmnn1 (FIG. 6B) strains carrying plasmid encoding Kre2-GnTI fusion and Mnn2-GnTII fusion under the control of the galactose inducible GAL1-10 promoter. Cells were induced for 24 hours with 2% galactose. Induction of Kre2-GnTI fusion yields additional peak at m/z of 1661 representing the complex N-glycan GlcNAc2Man3 (Gn2M3). Peaks at m/z of 1371 (M3), of 1375 (M4), of 1579 (M5), at m/z of 1620 (GnM4), and at m/z of 1661 (Gn2M3) are present. In Δalg11Δalg3Δmnn1 strain cell the peak at m/z of 1597 (M5) is strongly reduced as shown in FIG. 2C

FIG. 7 depicts Western blot analysis of Δalg11Δalg3 (FIG. 7A) and Δalg11Δalg3Δmnn1 cell extracts (FIG. 7B) expressing Kre2-GnTI fusion under control of two different galactose inducible promoters (GALs and GAL1). Cells were induced with galactose for 0, 2 and 4 hours. Cell extracts were probed with anti-Flag antibody in order to detect Flag-tagged Kre2-GnTI.

FIG. 8 depicts Western blot analysis of Δalg11Δalg3 and Δalg11Δalg3Δmnn1 cell extracts expressing Kre2-GnTI fusion and Mnn2-GnTII fusion under control of the galactose inducible promoter GAL1-10. Cells were induced with galactose. Samples were harvested after 0, 2, 4 and 20 hours. Cell extracts were probed with anti-Flag antibody in order to detect Flag-tagged Kre2-GnTI fusion and Mnn2-GnTII fusion.

FIGS. 9 and 10 depict vector maps of vectors for expression of heterologous glycosyltransferases. Expression of Kre2-GnTI is driven by Gal1 promoter (FIG. 9). Coexpression of Kre2-GnTI and Mnn2-GnTII is under control of bidirectional Gal1-10 promoter. Expression of both genes is inducible by galactose.

FIG. 11 depicts vector maps of vectors for expression of heterologous galactosyltransferase. Expression of Mnn2-GalT and Mnn2-Gal10-GalT is driven by a galactose-inducible Gal1 promoter.

FIG. 12 depicts Western blot analysis expression of hGnTI and hGnTII under the control of galactose inducible promoter GAL1-10 in Δalg11Δalg3 double mutant cells and expression of hGalT or Gal10-hGalT under control of GAL1 galactose inducible promoter in Δalg11Δalg3 double mutant cells. Cells were grown in raffinose containing minimal media supplemented by 1 mol/l sorbitol, and induced with galactose for 30 hours. Immunoblot analysis of the cell extracts expressing different Golgi glycosyl transferases as indicated, using anti-Flag antibody. All the glycosyl transferases were C-terminally Flag-tagged. (vec=empty vector)

FIG. 13 depicts MALDI-TOF MS spectra of permethylated N-glycans isolated from cell wall proteins of Δalg3Δalg11 yeast mutant strain expressing hGnTI, hGnTII, and hGalT with the epimerase from S. pombe (GAL10-GalT; A) and of Δalg3 Δalg11 yeast mutant strain expressing hGnTI, hGnTII, and hGalT (B). The individual N-glycan peaks are annotated above the respective peaks, being Man3GlcNAc2 (M3) to Man6GlcNAc2 (M6). In addition to mannose each indicated structure contains the two proximal GlcNAc (Gn) residues of the eukaryotic N-glycan core structure. The peaks at m/z 1171.7 represent M3, at m/z 1375.8 M4, at m/z of 1579.9 M5, and at m/z 1784 M6. The peaks at m/z 1661.8 represent Gn2M3, at m/z 1620.9 could represent either GnM4 or GalGnM3, and at m/z 2070.2 represent Gal2Gn2M3.

FIG. 14 depicts a result of whole cell ELISA of Δalg3Δalg11 yeast double mutant cells using CGL2 lectin. The Δalg3Δalg11 double mutant strain containing empty vector (vec), or containing plasmids for inducible expression of GnTI and GnTII, or GnTI, GnTII, and GalT without or with the Gal10 epimerase were used as indicated. In two streptavidin background controls (neg1, neg 2) induced cells were used expressing GnTI, GnTII and GalT (0.5 and 0.8 OD of the cells) and incubated only with streptavidin-HRP not with the biotinylated lectin.

FIG. 15 depicts MALDI-TOF MS spectra of the 2-AB labeled N-glycans isolated from cell wall proteins of Δalg3Δalg11 yeast mutant strain with empty vector (A), and of Δalg3Δalg11 yeast mutant strain expressing hGnTI, hGnTII, and hGalT with the epimerase from S. pombe (Gal10-GalT) without enzyme treatment (B), and with beta-galactosidase treatment at 37° C. (C). The two peaks representing potential presence of galactose (at m/z 1621.6 and m/z 1783.7) disappeared after beta-galactosidase treatment. Instead, the peak representing Gn2M3 at m/z 1459.6 is increased, confirming the presence of terminal galactose on the N-glycans.

FIG. 16 depicts MS/MS MALDI-TOF spectra of the permethylated N-linked glycans of the Δalg11Δalg3 double mutant strain expressing hGnTI and hGnTII (A), and of the Δalg11Δalg3 double mutant strain expressing hGnTI, hGnTII, and hGalT (B-D). The characteristic ionic fragments containing a terminal (non-reducing end) sugar units are indicated by B, C, and D. The Y fragment represents ions containing the reducing sugar unit.

FIG. 17 depicts the result of a purification of secreted acid phosphatase co-expressing GnTI, GnTII and AP. The strain was transformed with a plasmid carrying the pho5 gene under the control of GPD promoter expressing a C-terminally His-tagged AP and a plasmid pAX428 for galactose inducible expression of GnTI and GnTII. Cells were grown in minimal media and expression was induced by addition of galactose. Expression of GnTI and GnTII was verified using Western blot using anti-Flag antibody (a-Flag) (upper left panel). Expression of His-tagged acid phosphatase (AP) was verified using anti-His antibody (a-His) immunobloting (lower left panel). Secreted AP from 150 ml batch culture was purified using affinity chromatography and the fractions analyzed using SDS-PAGE and silver staining (right panel). AP was eluted from Ni-NTA column at an imidazole concentration of 100 mmol/l (L=load; F_{T}=flow through; W=wash at 10 mmol/l imidazole; 1 to 6=eluted fractions at 100 mol/l imidazole).

FIG. 18 depicts MALDI-TOF MS spectra of permethylated N-glycans released from purified acid phosphatase of Δalg3Δalg11 yeast mutant strain expressing acid phosphatase and GnTI and GnTII under control of a galactose inducible promoter. A) non-induced control culture, B) galactose-induced culture of Δalg3Δalg11 yeast mutant strain expressing GnTI and GnTII in addition to acid phosphatase (AP). M4 and M5 indicate Man4GlcNAc2 and Man5GlcNAc2 N-glycan structures, respectively. The complex target structure GlcNAc2Man3GlcNAc2 is detected at an m/z of 1662.16 (indicated as Gn2M3) in FIG. 18B but is absent in FIG. 18A.

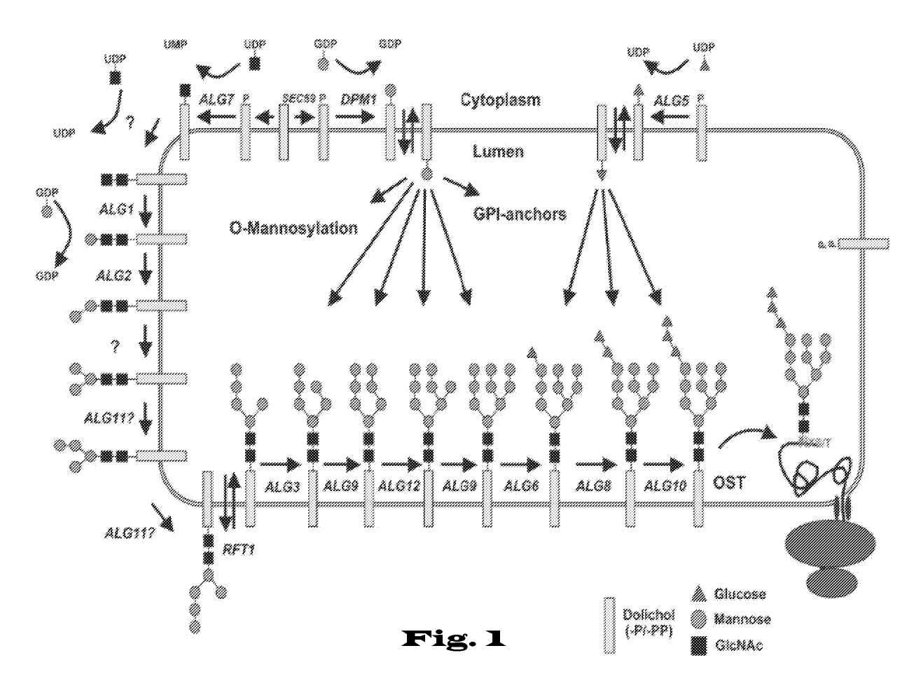

### EXAMPLES

1. Generation of Δalg3 Δalg11 Strain

The entire ALG11 open reading frame was replaced in wild-type cells SS328×SS330 by integration of a PCR product containing the S. cerevisiae HIS3 locus. The resulting strain (MATa/α ade2-201/ade2-201 ura3-52/ura3-52 his36200/his36200 tyr1/+ lys2-801/+Δalg11::HIS3/+) was sporulated and tetrads were dissected to obtain a Δalg11 haploid strain (MATα ade2-201 ura3-52 his36200 Δalg11::HIS3). The Δalg11 haploid strain was mated with a Δalg3 strain (MATa Δalg3::HIS3 ade2-101 his36200 lys2-801 ura3-52). The resulting diploid strain (MATa/α ade2-201/ade2-201 ura3-52/ura3-52 his-3Δ200/his36200 lys2-801/+Δalg3::HIS3 Δalg11::HIS3/+) was sporulated and tetrads were dissected on YPD plates containing 1 mol/l sorbitol to obtain the haploid Δalg3Δalg11 double mutant strain (MATα ade2-101 ura3-52 his36200 lys2-801 Δalg3::HIS3).

2. Generation of Δalg11 Δalg3 Δmnn1 triple mutant strain

The MNN1 locus was deleted in yeast wild-type cells (SS330) using a PCR product containing the S. cerevisiae HIS3MX cassette. The Δmnn1 deletion was combined with the Δalg3 deletion strain (see above) by crossing the two mutant strains. The resulting diploid strain was then sporulated and tetrads were dissected. A haploid strain carrying both mnn1 and alg3 deletions was selected. The deletions were further confirmed by PCR analysis.

The constructed haploid double mutant strain Δalg34 nm n1 was then crossed with Δalg3Δalg11 double mutant strain (MATα ade2-101 ura3-52 his38200 lys2-801 Δalg3::HIS3 Δalg11::HIS3). The resulting diploid strain was sporulated and tetrads were dissected. A haploid triple mutant strain containing all three deletions alg11, alg3, and mnn1 was selected and the deletions were further confirmed by PCR analysis.

### 3. Expression of GlcNAc Transferases in Yeast

### 3.1 Expression of Human GnTI

Human GnTI (hGnTI), mannosyl (alpha-1,3-)-glycoprotein beta-1,2-N-acetylglucosaminyl transferase, is a medial Golgi enzyme which is essential for the synthesis of hybrid and complex N-glycans from high mannose type N-glycans. The human GnTI is a type II transmembrane protein encoded by a single exon. The hGnTI consists of an N-terminal cytoplasmic tail and a transmembrane domain followed by a so called stem domain, which together are responsible for the proper localization and protein interactions. A large C-terminal catalytic domain is located in the Golgi lumen.

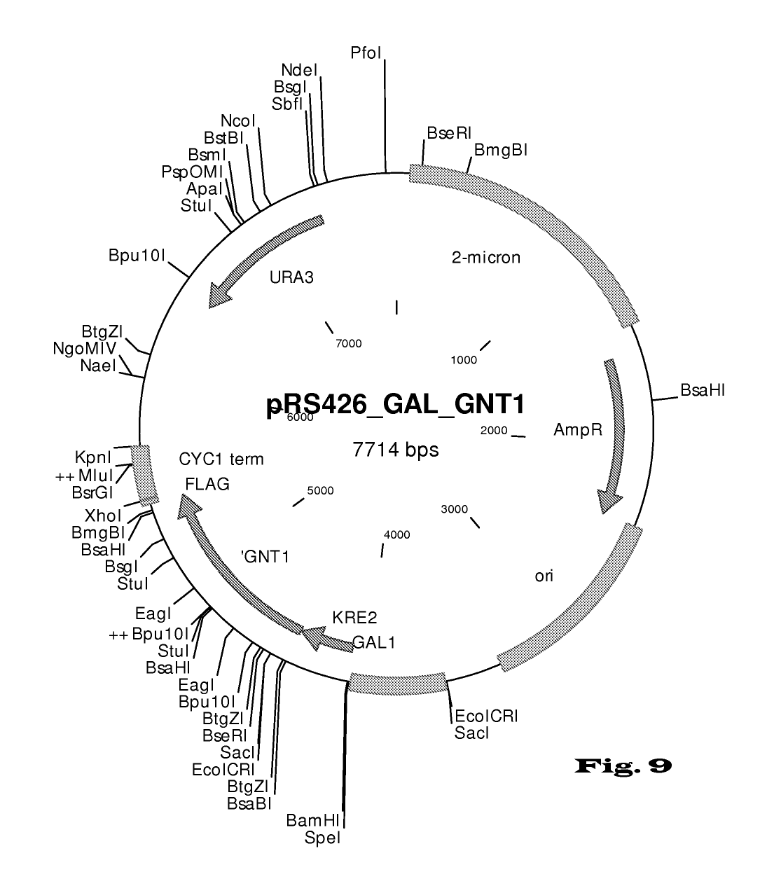

In order to express the hGnTI in S. cerevisiae we cloned the hGnTI in a high copy number plasmid under the inducible GALs and GAL1 promoters, with a FLAG tag at the C-terminus (FIG. 9). For proper localization of the enzyme to the Golgi the N-terminal transmembrane and stem domain of the yeast Kre2p was fused to the catalytically active domain of the hGnTI yielding Kre2-GnTI fusion protein. The yeast Kre2p is a typical type II transmembrane protein localized to the early Golgi with mannosyl transferase activity.

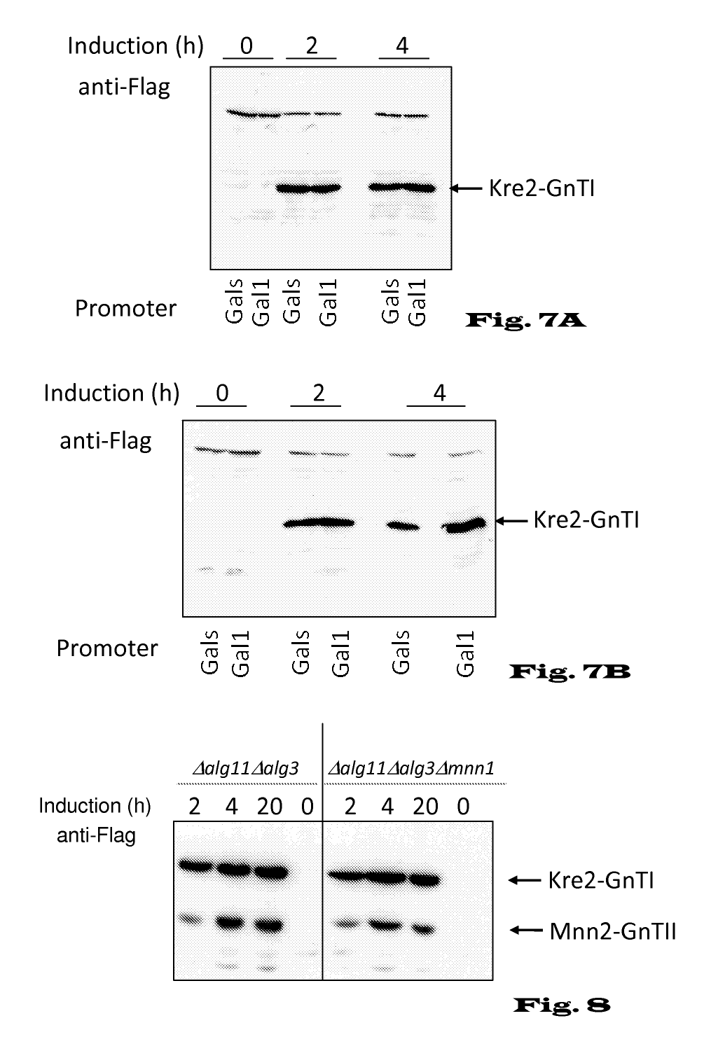

Both, the Δalg11 Δalg3 double mutant and Δalg11 Δalg3Δmnn1 triple mutant strains were transformed with the hGnTI encoding plasmid. The expression of the Kre2-GnTI fusion was confirmed by immunobloting analysis using anti-FLAG antibody (FIG. 7).

### 3. 2 Expression of Human GnTII

The product of the human GnTII is a Golgi enzyme catalyzing the addition of the second alpha-1,2-linked GlcNAc to the alpha-1,6-mannose. The enzyme has the typical glycosyl transferase domains: a short N-terminal cytoplasmic domain, a hydrophobic non-cleavable signal-anchor domain, and a C-terminal catalytic domain. The coding region of this gene is intronless.

To localize the hGnTII to the Golgi, the N-terminal transmembrane and stem domain of the yeast Mnn2p was fused to the catalytically active domain of the hGnTII yielding Mnn2-GnTII fusion protein. The yeast Mnn2p is a typical type II transmembrane protein localized to the early Golgi with mannosyl transferase activity.

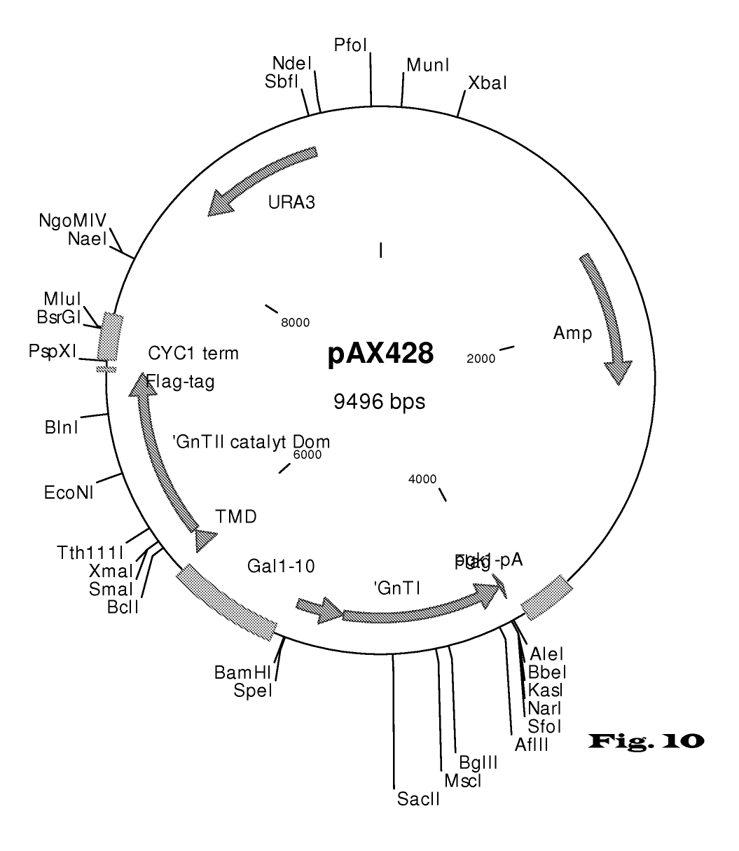

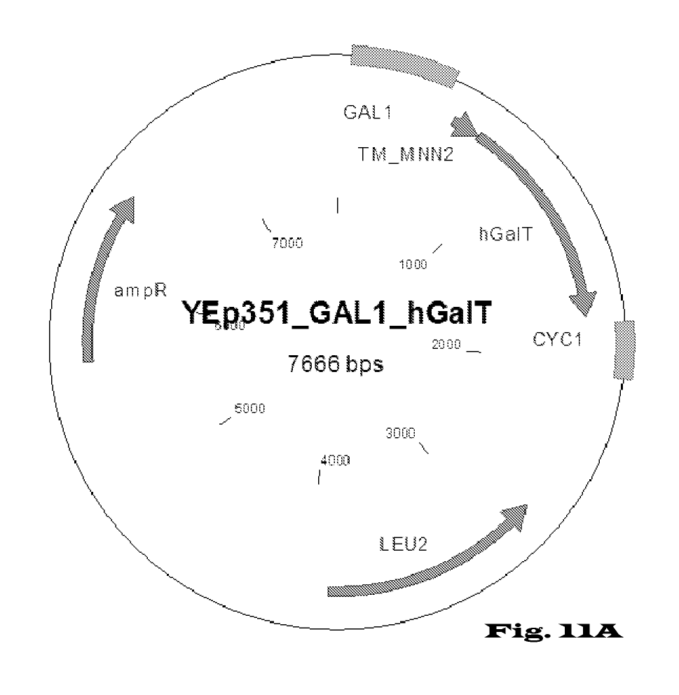

In order to co-express the Kre2-GnTI fusion and Mnn2-GnTII in S. cerevisiae the inducible GAL1-GAL10 promoter was used in a high copy number plasmid (FIG. 10). The Kre2-GnTI fusion was cloned under the control of the GAL1 promoter and Mnn2-GnTII under the control of the GAL10 promoter. Both Kre2-GnTI and Mnn2-GnTII fusion proteins were C-terminally Flag tagged.

The Δalg11 Δalg3 double mutant and Δalg11 Δalg3Δmnn1 triple mutant strains were transformed with the plasmid encoding Kre2-GnTI fusion and Mnn2-GnTII. The co-expression of the Kre2-GnTI and Mnn2-GnTII fusion proteins was confirmed by immunobloting analysis using anti-Flag antibody (FIG. 8).

### 4. MALDI-TOF MS

For analysis of N-glycans from cell wall proteins, cells were broken in 10 mmol/l Tris using glass beads and the insoluble cell wall fractions was reduced in a buffer containing 2 mol/l thiourea, 7 mol/l urea, 2% SDS 50 mmol/l Tris, pH 8.0 and 10 mmol/l DTT. Alkylation was performed in the identical buffer containing 25 mmol/l iodoacetamid for 1 hour at 37° C. under vigorous shaking. The cell wall fraction was collected by centrifugation and the resulting pellet washed in 50 mmol/NH4CO3.

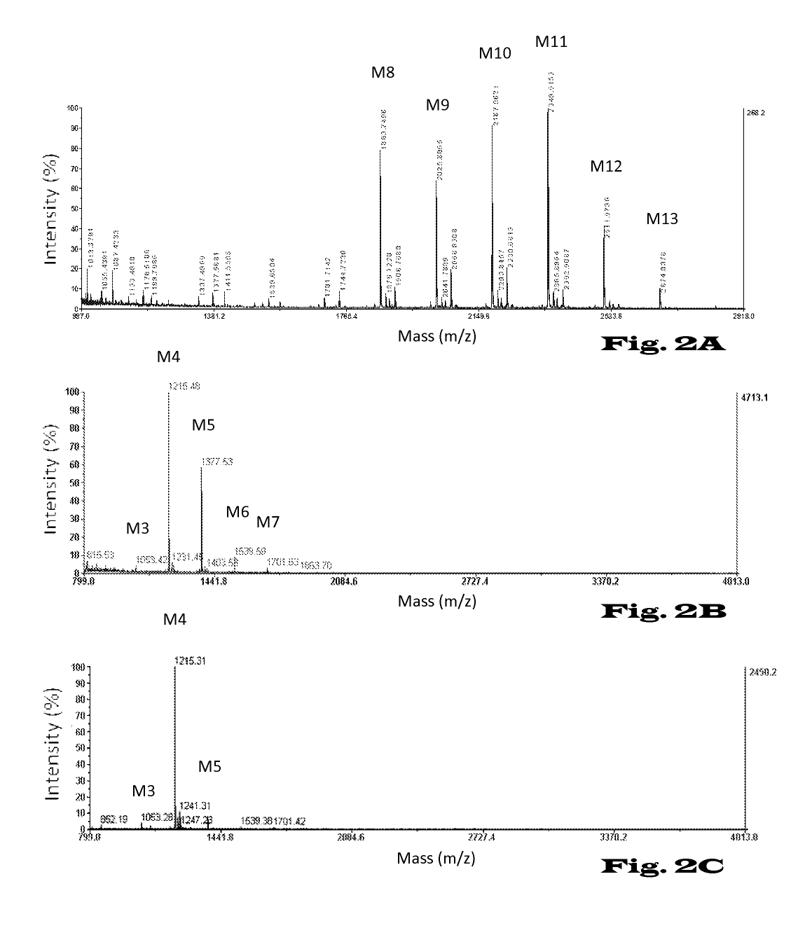

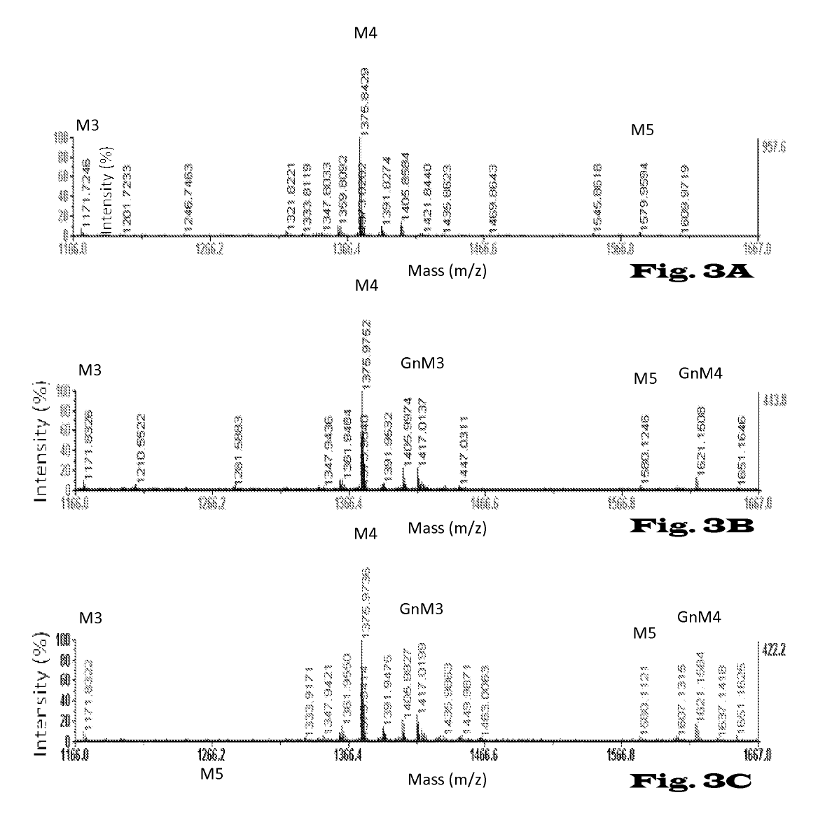

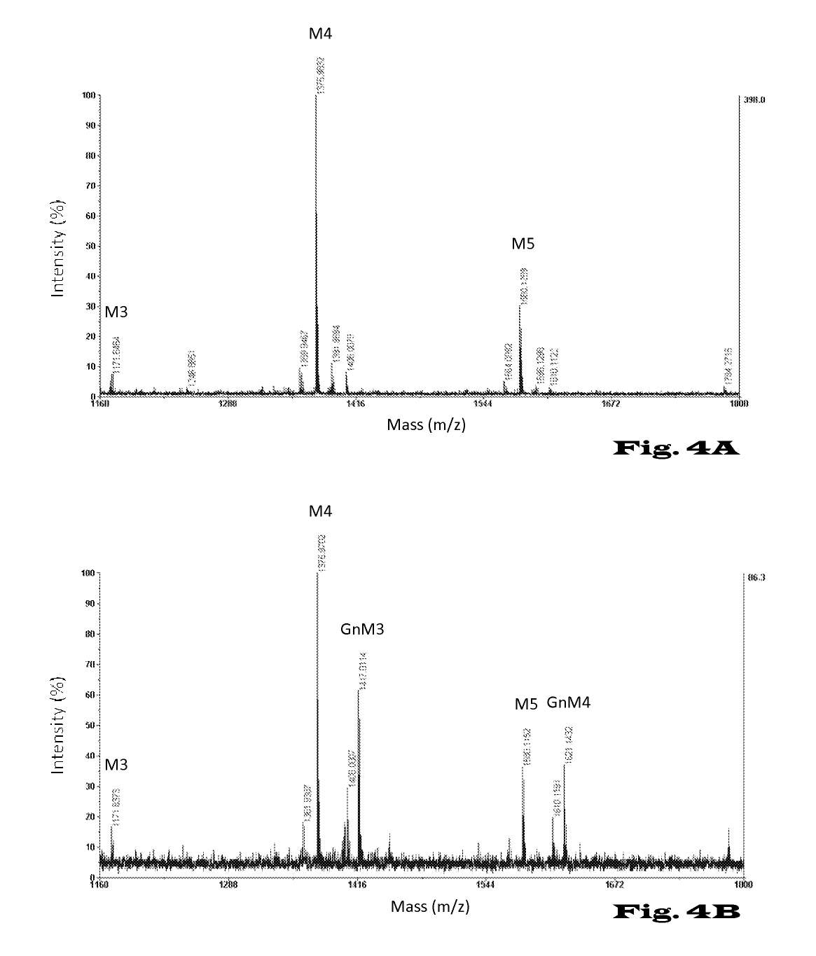

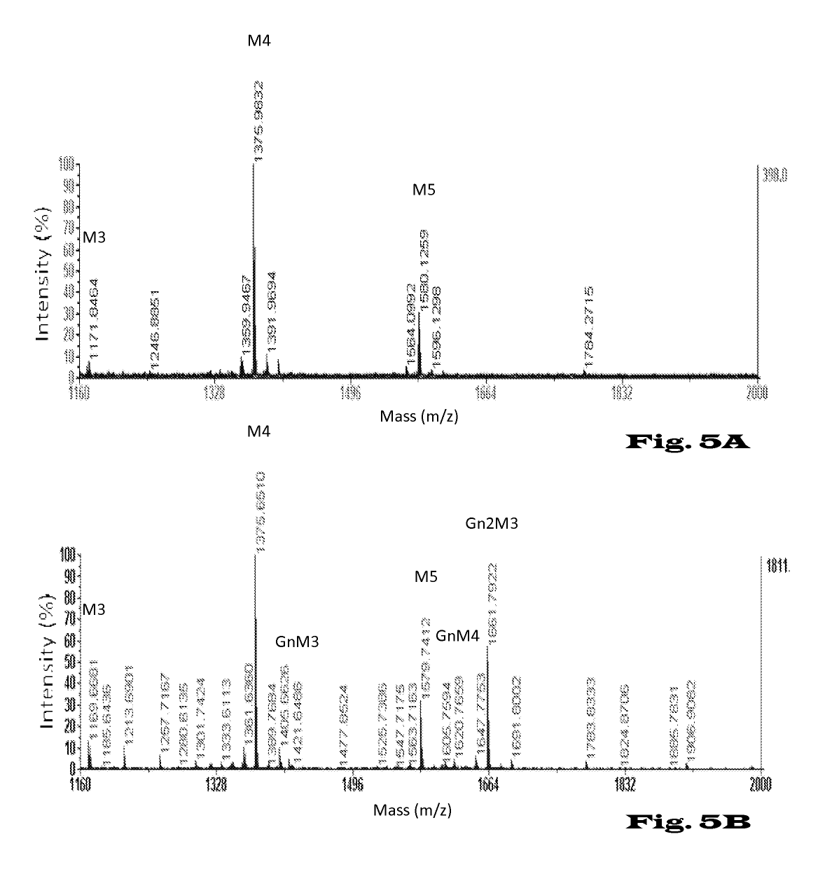

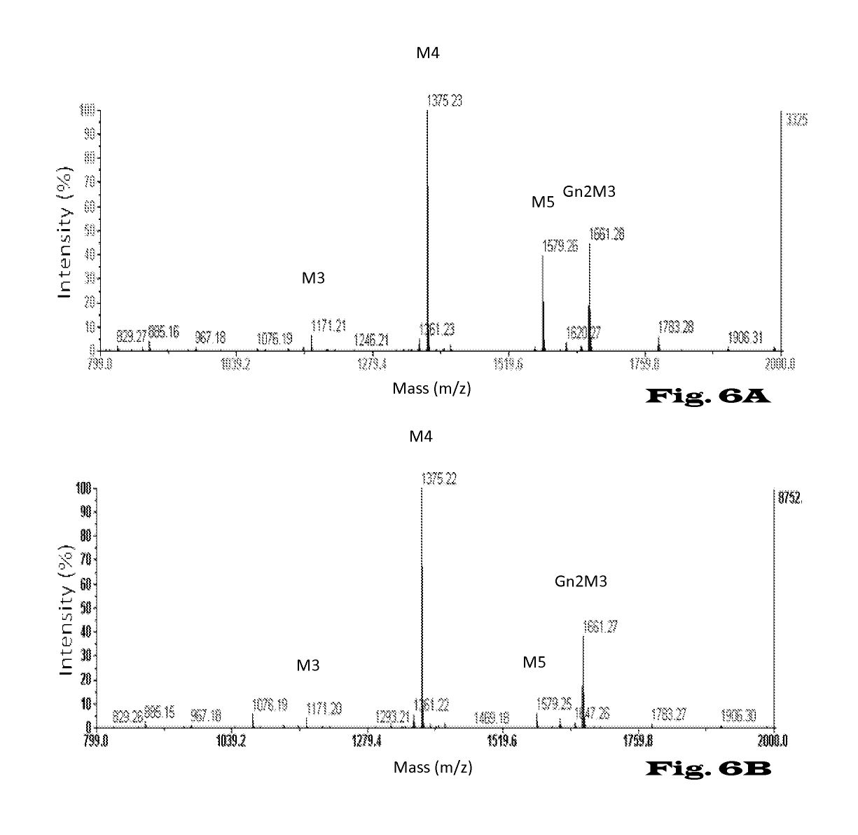

N-glycan were released overnight at 37° C. using 1 μl PNGase F in a buffer containing 1× denaturation buffer, 50 mmol/l phosphate buffer, pH 7.5, and 1% NP-40. N-glycans were purified via C18 and Carbon columns and the eluate containing the N glycans evaporated. N-glycans were labeled with 2-aminobenzamide or permethylated. Mass spectra of purified N-glycan preparation were acquired using an Autoflex MALDI-TOF MS (Bruker Daltonics, Fällanden, Switzerland) in positive ion mode and operated in reflector mode. An m/z range of 800-3000 was measured (FIGS. 2 to 6).

### 5. Growth Conditions for Expression of GnTI and GnTII

The Δalg11Δalg3 double mutant and Δalg11Δalg3Δmnn1 triple mutant strains carrying the plasmid encoding Kre2-GnTI and Mnn2-GnTII were grown in synthetic minimal medium lacking uracil (SD-Ura) to mid-log phase at 26° C. to an OD600 nmol/l of 1. The SD-Ura medium contained 2% raffinose and 1 mol/l sorbitol. Cells were then induced by exchanging the medium to SD-Ura containing 2% galactose and 1 mol/l sorbitol, and grown for the indicated times.

### 6. Expression of the Human GalT

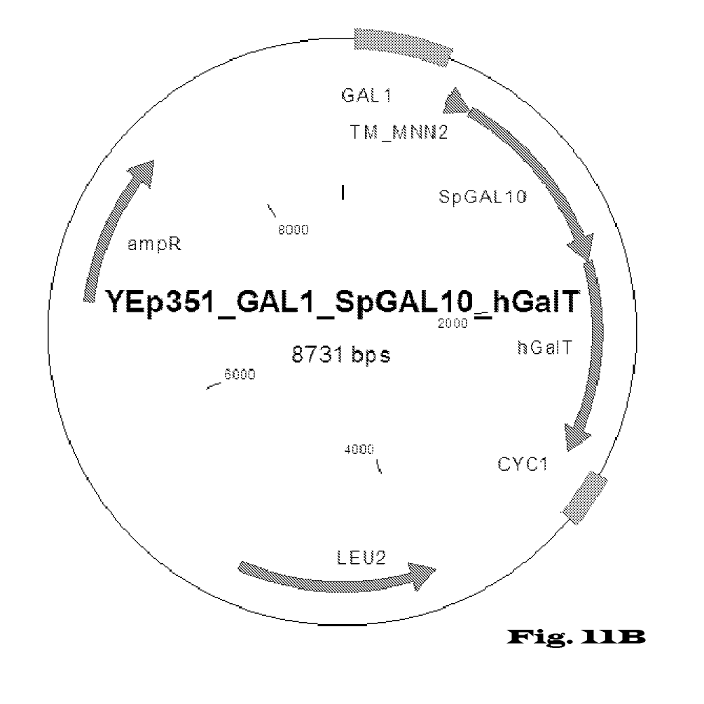

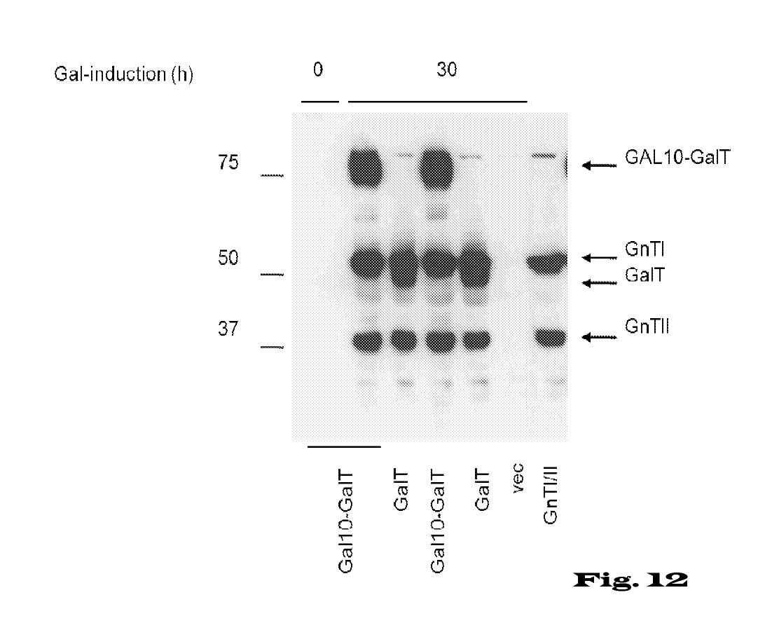

Higher complexity of humanization of N-linked glycans in the yeast strains requires expressions of further glycosyl transferases in the Golgi. Since the GlcNAc-transferases (hGnTI and hGnTII) can be successfully expressed under the galactose inducible GAL1-10 promoter, and two GlcNAcs can be transferred onto the M3 glycans (see above) galactosyl transferase was expressed in the Golgi in addition: UDP-Gal:betaGlcNAc beta 1,4-galactosyl transferase is a type II membrane bound protein localized to the Golgi encoded by one of seven beta-1,4-galactosyl transferase (beta4GalT) genes. These type II membrane proteins have an N-terminal hydrophobic signal sequence for Golgi localization which remains uncleaved. They all transfer galactose in a beta 1,4 linkage to similar acceptor sugars GlcNAc, Glc, and Xyl. To express the human GalT (hGalT) in the yeast Golgi, transmembrane and stem domains of S. cerevisiae Mnn2p were fused to the catalytically active domain of hGalT. Mnn2p is a type II Golgi membrane protein with mannosyl transferase activity which herein is used for proper localization of the hGnTI in the Golgi. A double or a triple fusion of the catalytically active domain of hGalT was used: In the double fusion, the catalytic domain of hGalT was fused to the Golgi localization domain of the yeast Mnn2p in the triple fusion also the full length UDP-galactose 4-epimerase from S. pombe which is encoded by GAL10 gene is included. Stem and transmembrane domains of the Mnn2p were amplified from the yeast genomic DNA. The catalytic domain of hGalT from the human GalT cDNA, and the full length of SpGAL10 from its cDNA were amplified. These fusion proteins were cloned under the yeast galactose inducible GAL1 promoter on two different high copy number plasmids pRS425 and YEp351 with LEU2 gene markers. hGalT and Gal10-hGalT were both C-terminally Flag-tagged (FIG. 11). The Δalg11Δalg3 double mutant strain was transformed with the plasmid encoding hGnTI and hGnTII under the GAL1-10 promoter. This plasmid contained a URA3 gene marker. The resulting strain was then transformed with the hGalT or SpGal10-hGalT containing plasmids with a LEU2 gene marker. The expressions of hGnTI, hGnTII, and hGalT with and without the epimerase fusion were confirmed by immunoblot analysis using anti-Flag antibody (FIG. 12).

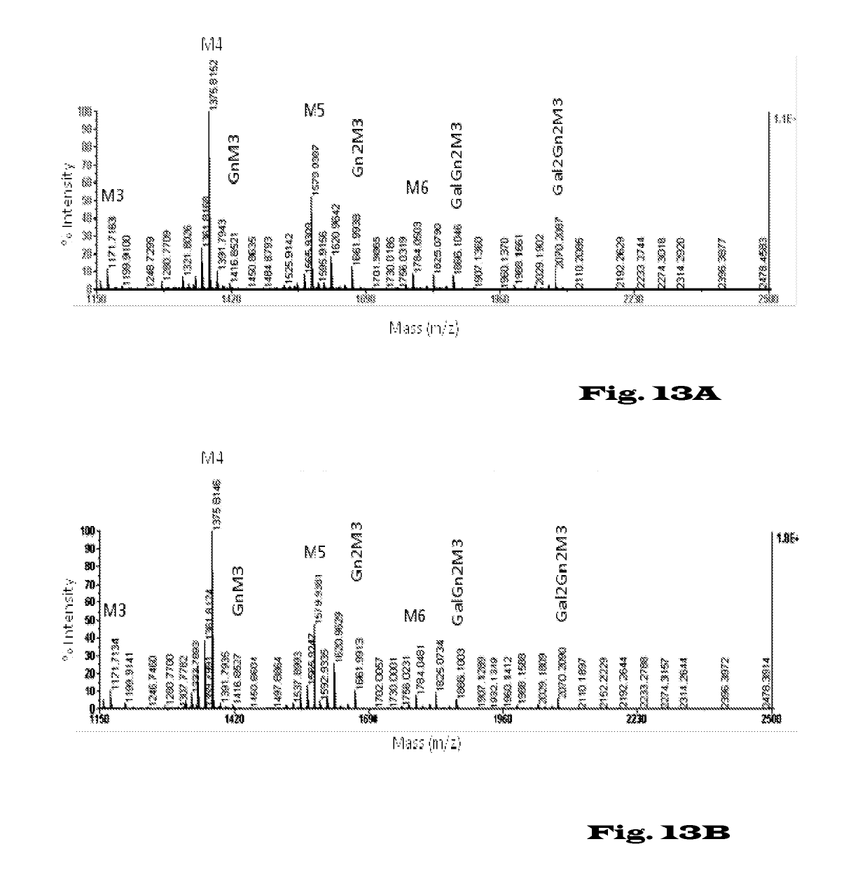

In order to know whether this enzyme was active in vivo, cell wall N-linked oligosaccarides of these strains were released from the cell wall proteins by PNGase-F enzyme, after reduction and alkylation they were purified. The N-linked glycans were permethylated and analyzed by MALDI-TOF MS analysis. The MS profile confirmed the transfer of one and two hexoses onto the GlcNAc2Man3GlcNAc2 glycan structure, respectively (FIG. 13).

To confirm the presence of terminal galactose to be the additional hexose(s) on GlcNAc2Man3, (1) a whole cell ELISA using CGL2 lectin, (2) a specific cleavage of the terminal galactose by a beta-galactosidase enzyme and (3) tandem mass spectrometry analysis on the purified N-linked glycans were employed.

### a) Whole Cell ELISA Using CGL2 Lectin

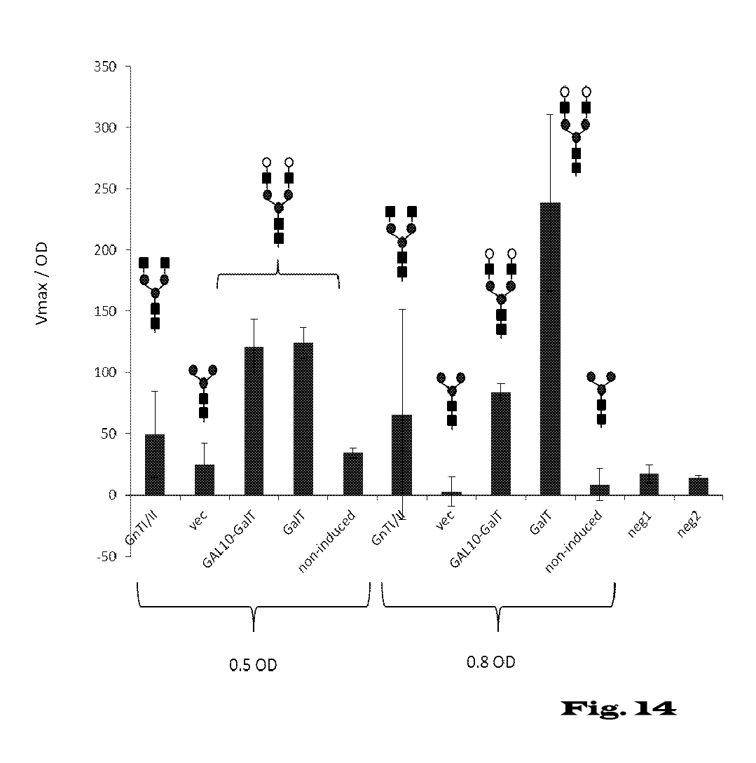

A whole cell ELISA was performed using biotinylated CGL2 lectin. CGL2 is a galactose binding lectin that binds beta-galactosides such as lactose. Briefly, cells carrying empty vector or plasmids expressing hGnTI and hGnTII, or hGnTI, hGnTII, and hGalT with or without the Gal10 epimerase were grown to OD600 of 1 in minimal medium containing 1 mol/l sorbitol (20 ml in shake flasks). Cells were then diluted to OD600 of 0.5 and the medium was exchanged with the galactose containing minimal medium for galactose-inducing expression of the transferases and grown for 24 to 36 hours till OD600 of 1 was reached. A volume corresponding to 0.5 or 0.8 OD600 of the cells was harvested and washed with PBS. galactose structure was assayed with biotinylated CGL2, added at a final concentration of 3 μg/mL on a rotary wheel for 1 hour at 4° C. Cells were washed twice with PBS buffer, and incubated with streptavidin-HRP at a final concentration of 1 μg/mL for 1 hour at 4° C. Cells were then washed twice with PBS buffer and pelleted in 1 ml 70 mmol/l citrate phosphate buffer pH 4.2. Cell suspensions (150 μl per well) were distributed in 96 well plates for triplicate measurements. OD600 was measured with SpectraMax Plus384 (Molecular Devices) before adding 50 μl of freshly prepared 4×ABTS buffer. Vmax kinetics was monitored using “Kinetic ELISA with HRP and ABTS” of the program SoftMax Pro 4.8 (Molecular Devices) at 405 nm for 30 minutes (FIG. 14). The assay was validated by different negative controls: (1) The double mutant strain carrying empty vector with galactose induction; (2) the strain containing hGnTI, hGnTII, and hGalT without galactose induction, and (3) two negative controls for the ELISA assay (background controls) in which cells were incubated only with streptavidin-HRP but not with the biotinylated lectin.

The Vmax values of the cells expressing GnTI, GnTII, and GalT or GnTI, GnTII, and Gal10-GalT after galactose induction were increased compared to the cells carrying only empty vector or cells expressing only hGnTI and hGnTII.

### b) Beta-Galactosidase Treatment

To further analyze the additional hexoses on GlcNAc2Man3 N-linked glycan structures, the cell wall N-glycans were treated with beta-galactosidase enzyme which hydrolyzes beta-1,4- and beta-1,6-linkages and also beta-1,3 but slower than the first two linkages.

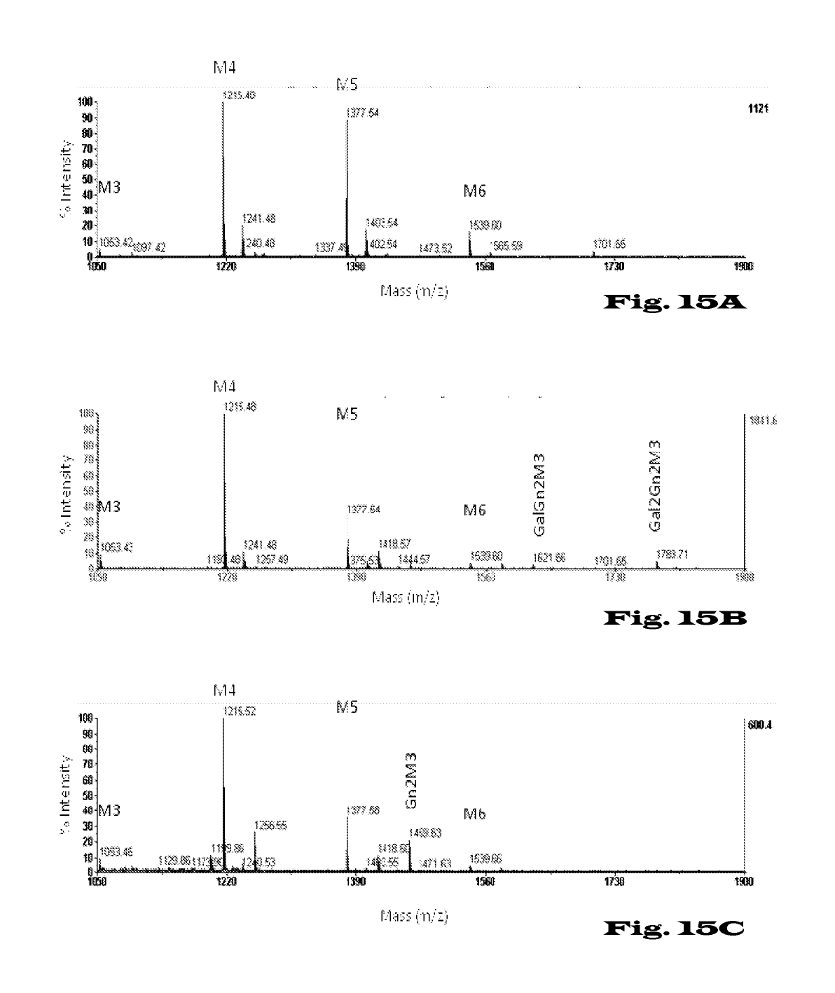

Cell wall N-linked oligosaccharides were released from the cell wall proteins by PNGase-F enzyme digestion, after reduction and alkylation they were labeled with 2-AB. The purified N-glycans were then incubated with beta-galactosidase enzyme at 37° C. overnight. The cell wall N-linked glycans were analyzed by MALDI-TOF MS analysis. The MS analysis confirmed the removal of terminal galactose upon enzymatic treatment (FIG. 15). Moreover, the peak at m/z 1459 representing GlcNAc2Man3 was increased upon enzymatic treatment, indicating the removal of galactose from GalGlcNAc2Man3 and Gal2GlcNAc2Man3 glycans.

### c) Tandem Mass Spectrometry of the N-Linked Glycans

Cell wall N-linked oligosaccharides of the strains expressing hGnTI, hGnTII, and hGalT were further analyzed by tandem mass spectrometry analysis. Tandem mass spectrometry known as MS/MS involves multiple steps of mass spectrometry selection, with some form of fragmentation occurring in between the stages. The MS/MS spectra of the cell wall N-linked oligosaccharides were obtained using collision induced decomposition (CID).

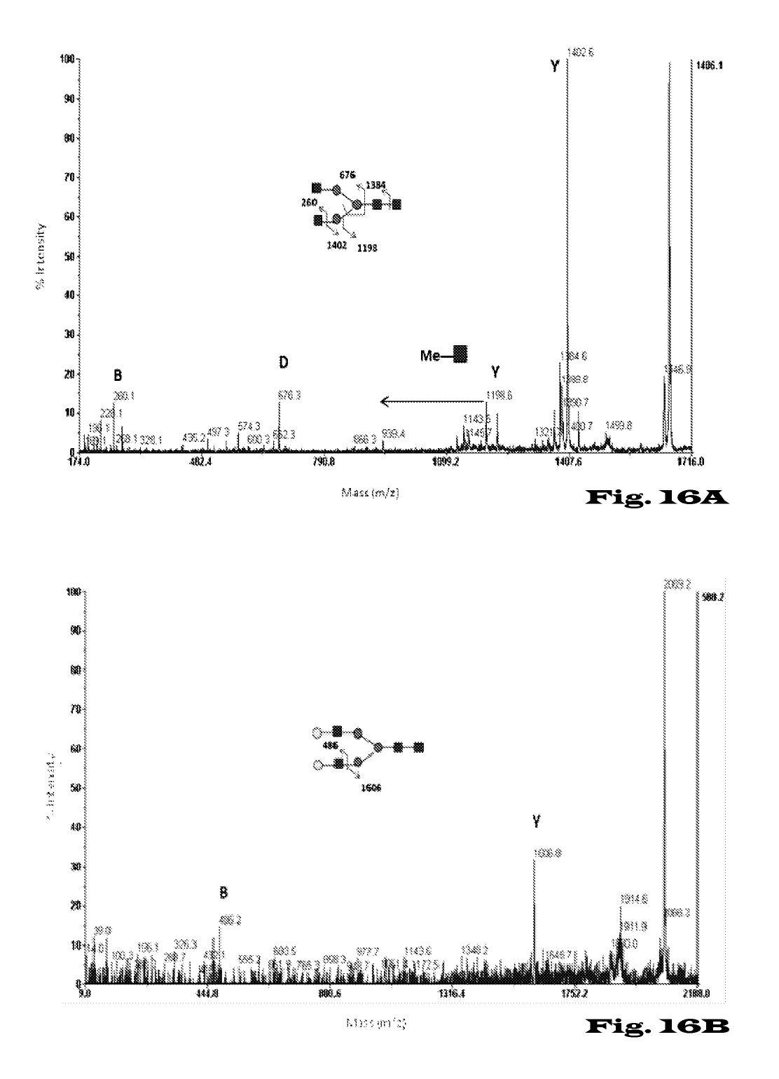

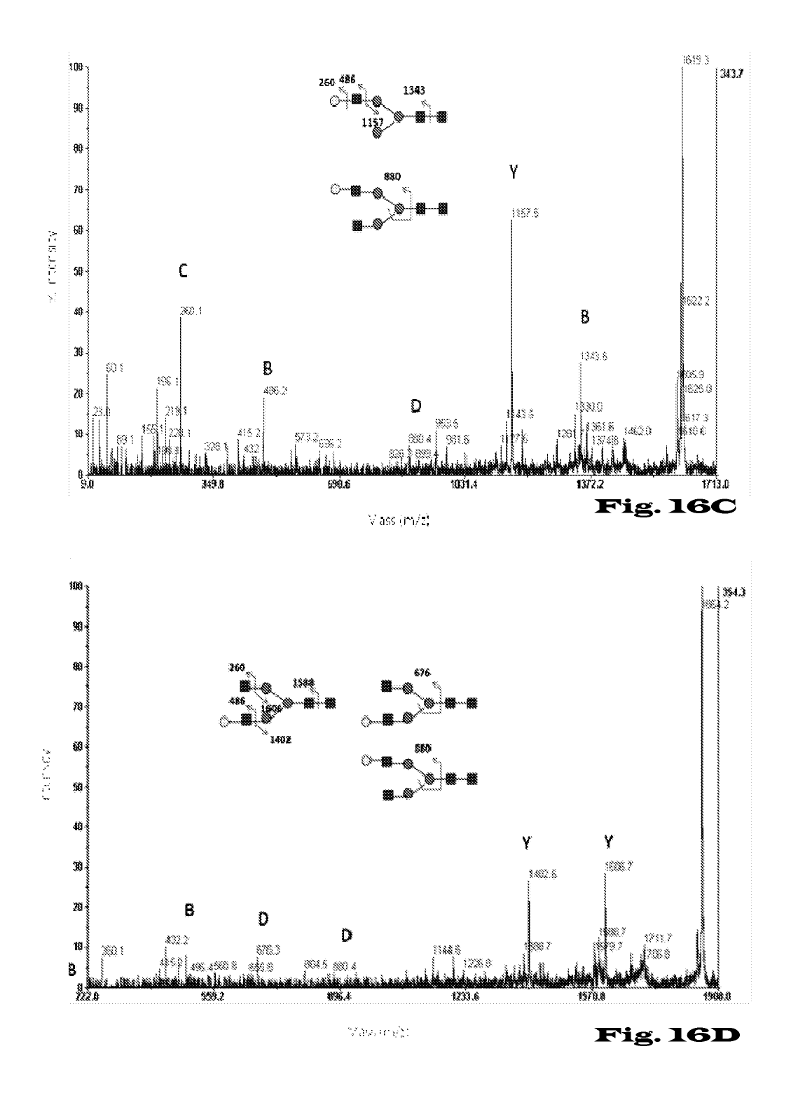

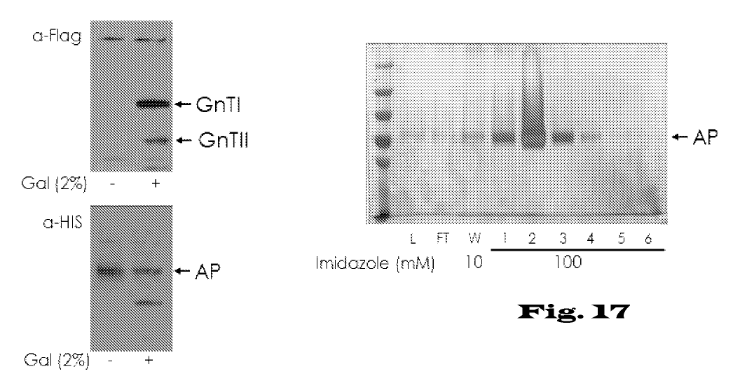

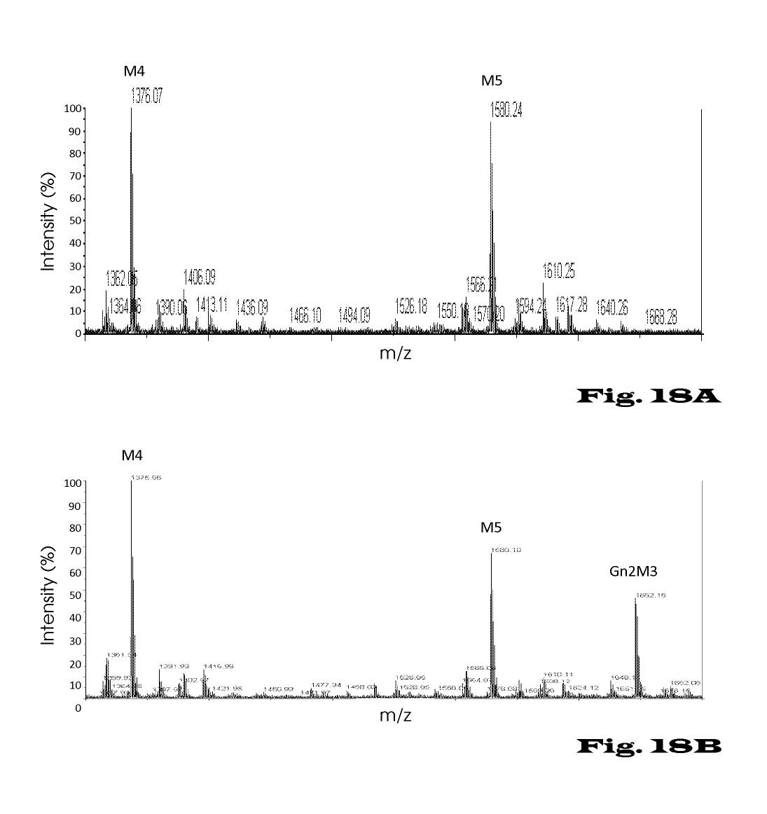

Fragmentation of the permethylated cell wall N-glycans of Δalg11 Δalg3 double mutant strain confirmed the expected sugar structures when hGnTI and hGnTII were expressed. The characteristic D-ion for the GlcNAc was detected at m/z 676 and also at m/z 260 (FIG. 16A). MALDI-MS/MS analysis of permethylated N-glycans isolated form Δalg11 Δalg3 double mutant strain expressing hGnTI, hGnTII, and hGalT (FIG. 16B-D), revealed the presence of the characteristic fragmentation ions for LacNAc with m/z 486 and m/z 260. The ion fragment at m/z 260 represented a hexose at the non-reducing end. Analysis of all the fragmentation ions from the parental glycans confirmed the expected N-linked glycan structures on the cell wall proteins isolated from the double mutant strain (FIG. 16).

From ELISA assay, beta-galactosidase treatment, and MS/MS analysis of the N-glycans we concluded that galactose was transferred onto the N-linked glycans of the Δalg11 Δalg3 double mutant strain by expressing the human GlcNAc-transferases and human galactosyl transferase.

### 7. Expression and Purification of N-Glycosylated Protein

The endogenous yeast acid phosphatase (AP) was used as a model protein to test complex glycosylation. The Δalg11Δalg3 double mutant strain carrying the plasmid encoding Kre2-GnTI and Kre2-GnTII under the control of the bidirectional galactose inducible promoter Gal1-10 strain was transformed with a plasmid carrying the pho5 gene encoding AP under the control of GPD promoter expressing a C-terminally His-tagged AP. Precultures were grown in minimal media containing 1% raffinose as carbon source. Cells were collected by centrifugation and resuspended in fresh media and expression was induced by addition of 2% galactose to the medium. A non-induced control culture was grown in the identical media as used for precultures. Secreted AP from 150 ml batch culture was purified using affinity chromatography. The culture supernatant was cleared by centrifugation at 15,000 g 4° C. for 15 minutes and was adjusted to 300 mmol/l NaCl, 10 mmol/l imidazole and 20 mmol/l Tris, pH 8.0. The supernatant was passed over a Ni-NTA agarose column (Qiagen) equilibrated with a buffer containing 300 mmol/l NaCl, 10 mmol/l imidazole and 20 mmol/l Tris, pH 8.0. The column was washed with 10 column volumes equilibration buffer. Fractions of 1 ml were eluted with a buffer containing 300 mmol/l NaCl, 100 mmol/l imidazole and 20 mmol/l Tris, pH 8.0. The fractions were analyzed using SDS-PAGE and silver staining.

N-glycans were released from purified AP using PNGaseF. N-glycans were purified using C18 and graphitized carbon columns. Purified N-glycans were permethylated and analyzed using an Autoflex MALDI-TOF MS (Bruker Daltonics, Fällanden, Switzerland).

1. A cell modified to
a) having suppressed, diminished or depleted ER-localized alpha-1,2-mannosyl transferase activity and ER-localized dolichyl phosphate-mannose glycolipid alpha-mannosyl transferase activity, and
b) expressing one or more nucleic acid molecule coding for a heterologous enzyme or catalytic domain thereof, selected from mannosyl (alpha-1,3-)-glycoprotein beta-1,2-N-acetylglucosaminyl transferase (GnTI) activity.

2. The cell of claim 1, which is a knock-out mutant of the genes alg11 and/or alg11 homologues and alg3 and/or alg3 homologues.

3. The cell of claim 1, further expressing one or more nucleic acid molecule coding for a heterologous enzyme or catalytic domain thereof, selected from mannosyl (alpha-1,6-)-glycoprotein beta-1,2-N-acetylglucosaminyl transferase (GnTII) activity.

4. The cell of claim 1, further expressing one or more nucleic acid molecule coding for a heterologous enzyme or catalytic domain thereof, selected from beta-N-acetylglucosaminyl glycopeptide beta-1,4-galactosyl transferase (GalT) activity.

5. The cells of claim 1, further modified to have suppressed, diminished or depleted Golgi-localized alpha-1,3-mannosyl transferase activity.

6. The cell of claim 5, which is a knock-out mutant of the gene mnn1 and/or mnn1 homologues.

7. The cell of claim 1, wherein the cell is further lacking or is having suppressed, diminished or depleted one or more further Golgi-localized mannosyl transferase activity.

8. The cell of claim 7, being a knock-out mutant of at least one gene selected from the group consisting of: och1, hoc1, mnn2, mnn5, mnn6, ktr6, mnn8, anp1, mnn9, mnn10, mnn11, mnt1, kre2, mnt2, mnt3, mnt4, ktr1, ktr2, ktr3, ktr4, ktr5, ktr7, van1, and yur1, and any homologues thereof.

9. The cell of claim 1, wherein the cell expresses one or more further Golgi-localized heterologous enzyme or catalytic domain thereof selected from the group consisting of:
beta-1,4-mannosyl-glycoprotein 4-beta-N-acetylglucosaminyl transferase (GnTIII);
mannosyl (alpha-1,3-)-glycoprotein beta-1,4-N-acetylglucosaminyl transferase (GnTIV);
mannosyl (alpha-1,6-)-glycoprotein beta-1,6-N-acetylglucosaminyl transferase (GnTV);
mannosyl (alpha-1,6-)-glycoprotein beta-1,4-N-acetylglucosaminyl transferase (GnTVI);
alpha (1,6) fucosyl transferase (FucT);
beta-galactoside alpha-2,6-sialyl transferase (ST);
UDP-N-acetylglucosamine 2-epimerase (NeuC);
sialic acid synthase (NeuB);
CMP-Neu5Ac synthetase;
N-acylneuraminate-9-phosphate synthase;
N-acylneuraminate-9-phosphatase;
UDP-N-acetylglucosamine transporter;
UDP-galactose transporter;
GDP-fucose transporter;
CMP-sialic acid transporter;
nucleotide diphosphatase;
GDP-D-mannose 4,6-dehydratase; and
GDP-4-keto-6-deoxy-D-mannose-3,5-epimerase-4-reductase.

10. The cell of claim 1, wherein the cell expresses one or more further Golgi-localized heterologous enzyme or catalytic domain thereof selected from the group consisting of:
beta-1,4-mannosyl-glycoprotein 4-beta-N-acetylglucosaminyl transferase (GnTIII);
mannosyl (alpha-1,3-)-glycoprotein beta-1,4-N-acetylglucosaminyl transferase (GnTIV);
mannosyl (alpha-1,6-)-glycoprotein beta-1,6-N-acetylglucosaminyl transferase (GnTV);
mannosyl (alpha-1,6-)-glycoprotein beta-1,4-N-acetylglucosaminyl transferase (GnTVI);
alpha (1,6) fucosyl transferase (FucT);
beta-galactoside alpha-2,6-sialyltransferase (ST);
UDP-N-acetylglucosamine 2-epimerase (NeuC);
sialic acid synthase (NeuB);
CMP-Neu5Ac synthetase;
N-acylneuraminate-9-phosphate synthase;
N-acylneuraminate-9-phosphatase;
UDP-N-acetylglucosamine transporter;
UDP-galactose transporter;
GDP-fucose transporter;
CMP-sialic acid transporter;
nucleotide diphosphatase;
GDP-D-mannose 4,6-dehydratase;
GDP-4-keto-6-deoxy-D-mannose-3,5-epimerase-4-reductase; and
UDP-glucose 4-epimerase/UDP-galactose 4-epimerase.

11. The cell of claim 1, wherein the cell is selected from a group consisting of: lower eukaryotic cells, including fungal cells, and higher eukaryotic cells including mammalian cells, plant cells, and insect cells.

12. The cell of claim 1, wherein the cell is further modified to express or produce at least one heterologous and/or recombinant protein as substrate for glycosylation.

13. A method for the production of a host cell capable of improved glycosylation of proteins, the method comprising:
diminishing or depleting in the cell ER-localized alpha-1,2-mannosyl transferase activity (Alg11-type);
diminishing or depleting in the cell ER-localized dolichyl phosphate-mannose glycolipid alpha-mannosyl transferase activity (Alg3-type); and
transforming the cell with at least one nucleic acid molecule coding for heterologous mannosyl (alpha-1,3-)-glycoprotein beta-1,2-N-acetylglucosaminyl transferase (GnTI) activity, such that the cell is able to express or overexpress said activity.

14. The method of claim 13, further comprising:
diminishing or depleting in the cell Golgi-localized alpha-1,3 mannosyl transferase activity (Mnn1);

15. The method of claim 13, further comprising:
transforming the cell with at least one nucleic acid molecule coding for heterologous mannosyl (alpha-1,6-)-glycoprotein beta-1,2-N-acetylglucosaminyl transferase (GnTII) activity, such that the cell is able to express or overexpress said activity.

16. The method of claim 13, further comprising:
transforming the cell with at least one nucleic acid molecule coding for heterologous beta-N-acetylglucosaminyl glycopeptide beta-1,4-galactosyl transferase (GalT) activity, such that the cell is able to express or overexpress said activity.

17. The method of claim 13, further comprising:
transforming the cell with at least one nucleic acid molecule coding for heterologous recombinant protein as the substrate for glycosylation, such that the cell is able to express or overexpress said protein.

18. An isolated host cell or a plurality thereof, produced according to the method of claim 13.

19. A method for the production of a glycoprotein or a glycoprotein composition, the method comprising:
providing a cell according to claim 1;
culturing the cell in a culture medium under conditions that allow the production of the glycoprotein or glycoprotein composition in the cell; and
if necessary, isolating the glycoprotein or glycoprotein composition from the cell and/or the culture medium.

20. A kit for producing a glycoprotein or glycoprotein composition comprising:
a cell according to claim 1; and
culture medium for culturing the cell so as to confer the production of the glycoprotein.

21. An isolated glycoprotein or glycoprotein composition, producible or produced by the cell, according to claim 1.

22. A pharmaceutical composition comprising the glycoprotein or glycoprotein composition according to claim 21 and at least one pharmaceutically acceptable carrier or adjuvant.
